(19) United States
(12) Patent Application Publication
(10) Pub. No.: US 2008/0069805 A1
(43) Pub. Date: Mar. 20, 2008

(54) Use of stem cells to cure genetic diseases in humans cure for sickle cell anemia

(76) Inventor: Bruce M. Williams, Gainesville, FL (US)

Correspondence Address: Bruce M. Williams, 100 NE 8TH Ave. #408, Gainesville, FL, 32601, US

(21) Appl. No.: 11/131,888

(22) Filed: May 18, 2005

(51) Int. Cl. A61K 35/12; A61P 7/00; C12N 5/00

(52) U.S. Cl. 424/93.21; 435/325

(57) ABSTRACT

Transplant of donor perfectly matched HLA hematopoietic stem cell to cure Sickle cell anemia and other anemia such as leukemia. Sterilized In vivo transplantation of clinically adequate quantities of antibiotic protected HLA vector/or insertion corrected chimera stem cells, and switching protein. Stem cells can be transfected for Hbg SS, and other proteins such as minor HLA type that may cause Graft versus host disease (GvHD) or Host versus Graft disease (HvGD Universal donor blood, Rh-negative of any HLA type can be corrected to perfectly match that of any recipient. Batch universal stem calls are grown and selectively transformed to a chimera stem cell. The chimera stem cells are incubated in a bio-reactor in growth medium also containing human growth and maturation promotion polypeptide factors. The harvest is then prepared for clinical use and transplantation into the matching recipient. Recipient's stem cells are transformed by transfection or insertion of the beta hemoglobin gene.

### REFERENCE CITED

U.S. Pat. No. 5,811,301 September 1998

### OTHER REFERENCES

Adzick, N. S. M.D., and Longaker, M. T. M.D. (1991) Fetal Wound Healing, Department of Surgery Unversity of California at San Francisco School of Medicine, san Francisco, Calif., Elsevier, N.Y., p-11-120.

Balinsky, B. I., Dr. Biol. Sc. (1970) Introductin to embryology, 3^{rd} ed. W. B. Saunders Company, Philadelphia, Pa.

Balzarini, J., Hernandez, A. I., Roche, P., Esnouf, R., Karrlson, A., Camarasa, M. J., Perez-Perez, M. J., (2003) Non-nucleoside inhibitors of mitochdrial thymidine kinase (TK-2) differentially inhibit the closely related herpes simplex virus type 1 TK and Drosophila melanogaster multifunctional deoxynucleoside kinase, Molecular pharmacology, vol. 63, num. 2, p-263-270.

Barrett, J. A., Katayoun Rezvani, Scott Solomon, Anne M. Dickinson, Xiao N. Wang, Gail Stark, Hannah Cullup, Mark Jarvis, Peter G. Middleton and Nelson Chao, (2003) New Developments in Allotransplant Immunology, Hematology, vol. 1, p-350.

Bone Marrow Tranplants, (1992) Allogenic bone marrow transplants, chapter 4, HLA system, HLA Matching, and HLA typing retrieved Jan. 16, 2005 from bmtnews.org.

Bornhauser, M. Thiede, C., Platzbecker, U., Jenke, A., Helwig, A. Plettig, R., Frieberg-Ricter, J., Rollig, C., Geissler, G., Lutterbech, K., Oelschlager, U., and Ehninger, G., (2001) Dose-reduced conditioning and allogeneic hematopoietic stem cell transplant from unrelated donors in 42 patients, Clinical cancer research, vol. 7, p-2254-2262, The American Association for Cancer Research.

Campbell, N. A., Reece, J. B., and Mitchell, L. G. (1999) Biology, Eukaryotic cell cycle, Adison Westley, Longman, Inc., 5^{th} ed., p-206-225.

Ibid. Chase-Griffith transduction of bacterial cells with viral DNA, p-278-280.

Ibid. Cell cycle, p-109.

Clark, B. E., and Thein, S. L. (2004) Molecular diagnosis of haemoglobin disorders, review, Clinical and laboratory haematology, vol. 26, issue 3, p-159, retrieved Jan. 30, 2005 from http://www.blackwell-synergy.com.

Dedov, V. N., Dedova, I. V., Nicholson, G. A., (2004) Equillibrium between cell division and apoptosis in immortal cells as an alternative to the G1 restriction mechanism in mammalian cells, Cell cycle, vol. 3, num. 4, p-491-495.

Fabry, O. I., Humphries, M. E., Pauliuk, R., Leboulch, P., Hoffman, R. Nag, R El. L., Eaves, C. (2004) expression of an anti-sickling beta globin in human erythroblasts derived from retrovirally transduced primitive normal and sickle cell disease hematopoietic cells, Experimental Hematology, vol. 32, num. 5, p-461-469.

Fernandez, M. N., Regidor, C. Cabera, R., Garcia-Marco, J. A., Fores, R., Sanjuan, Experimental Hematology, Cordblood forum, (2003) New concepts in cord blood transplantation, vol. 31, p 535-544.

Gibson, T., and Medawar, P. B. (1943), ( Leukocyte Chimerism linked with Tolerance), Journal of Anatomy, vol. 77, p-299-310.

Gruenert, D. C., Bruscia, E., Novelli, G., Colosimo, A., Dallapiccola, B., Sangiuolo, F., and Goncz, K., (2003) Sequence-specific modification of genomic DNA by small DNA fragments, Journal of clinical investigation, vol. 112, p-637-641.

Herzog, E. L., Chai, L., and Krause, D. S., (2003) Plasticity of marrow derived stem cells, Blood, Review in Translational hematology, the American Society of Hematology, vol. 102, num. 10.

Hosaka, K., Sato, K., Okamoto N., Kazami, A., Sato, K. (2004) Development of reconstituted embryos derived from somaaic cell nuclei in the rabbit, Human cell, vol. 17, num. 1, p-29-32.

Kohn, D. B., Satake, N. (2001) Immune responses and tolerance in gene therapy, Division of Research Immunology/B.M.T., he John Connell Gene Therapy Program, Children's hospital Los Angeles, Calif., University Southern California, Keck School of Medicine.

Li, Q., Peterson, K. R. Fang, X., Stammatoyannopoulos, G., (2002) Locus Control Region, Blood, vol. 100, p-3077-86.

Lefrere, F., Levy, V., Makke, J., Audat, F., Cavazzana-Calvo, M., Miclea, J., m. (2004) Successful peripheral blood stem cell harvesting with grandulocyte colonu-stimulating factor alone after previous mobilization failure, Hematologica, vol. 89, num. 12, P-1532-1534.

Mansour, et. Al. (1988) Neomycin resistant gene as a selection process, Nature, London, vol. 336, p-348.

Nienhuis, A. W., (1978) Hemoglobin switching new experimental model, Journal of Laborataoary and clinical Medicine, vol. 91, num. 6, -857-861.

Oh, I. H., Fabry, M. F., Humphries, R. K., Pawliuk, R., Leboulch, P., Hofman, R., Nagel, R. I., and Eaves C. (2004) Expression of an anti-sickle beta-globin in human erythroblasts derived from retrovirally transduced primitive normal and sickle disease Hematopoietic cells, Experimental Hematology, vol. 32, num. 5, p-461-469.

Roitt, Ivan, Brostoff, Johnathan and Male, David, (1985) Immunology, “Tolerance and Auto-immunity”, Chapters 12 and 23, Gower Medical Publishing, London, New, York.

Rood, P. M., Calafat, J., Von Dem Borne, A. E., Gerritsen, W. R., Van der schoot, C. E., (2000) Immortilization of bone marrow endothelial cells: Characterization of new cell line, European journal of clinical investigation, vol. 30, Number 7, p-618-629.

Rund, D., Dobkin, C., and Bamk, A., (1987) Regulated expression of amplified human beta-globin genes, Blood, vol. 70, num. 3, p-733-739.

Savitt, T. L., and Goldberg, M. F. (1989) -Herrrick's 1910 case report of sickle cell anemia, The rest of the story, The Journal of the American Medical Association, vol. 261, num. 2, p-266-271.

Scheuermann, M. O., Tajbakhsh, J., Kurz, A., Saracoglu, K., Eils, R., Lichter, P., (2004) Topology of genes and non-transcribed sequences in human interphase nuclei, Experimental cell biology, vol. 310, num. 2, p-266-279.

Schroeder, W. A. Ph. D., Jakway, J., Powars, D. M.D., (1972) Detection of hemoglobin S and C at birth: a rapid screening procedure by column chromatography, California Institute of Technology, Pasadena Calif.

Services and Resources provided (n.d.) The cell and gene bank retrieved Jan. 26, 2005 from

http: www.ihwg.org/shared/cbankover.htm

Shinar, D., Yoffe, O., Shani, N., and Yaffe, D. (1989)Regulated expression of muscle-specific genes introduced into mouse embryonic stem cells: inverse correlation with DNA Methylation, Differentiation, vol. 41, num. 2, p-116-126.

Stevens, A., Wang, Y., Bremer, K., Zhang, J., Hoepfner, R., Antonious, M., Schoenberg, D. R., and Maquat, L. E., (2002) Beta-globin mRNA decay in erythroid cells: UG site preferred endonucleolytic cleavage that is augmented by a premature termination condon, Proceedings of the National Academy of Science, vol. 99, issue 20, p-12741-12746.

Steward, S., (1992) The Bone Marrow Transplants, The Transplant, and the engraftment, chapter one, p-15. BMT Newsletter, HIGHLANDPARK, Ill.

Tuan, D., Biro, P. A., deRiel, J. K., Lazarus, H., and Forget, B. G., (1979) Restriction endonuclease mapping of the human gamma globin gene loci, Nucleic acids Research, vol. 6, num. 7, p-2519-2544.

United States Patent and Trademark Office. (November, 2006). PTO-EBT files. Retrived Nov. 2,2006 m from http://www.uspto.gov/.

Van Pelt, K., De Haan, G., Vellenga, F., and Daenen, S, M. (2005) Administration of low dose cytarabine result in immediate S-phase arrest and subsequent activation of cell cycling in murine stem cells, Experimental Hematology, vol. 33, num. 2, p-226-231.

Weaver, S., Haigwood, N. L., Hutchinson, C. A., III and Edgell, M. H. (1979) DNA Fragment of the Mus Musculus beta-globin haplotypes Hbbs, Proceedings of the National Academy of Science, USA, vol. 76, p-1385-1389.

Williams, B. M. (Mar. 28, 1956-2005) Unpublished personal research and theorectical papers, file (BMWI Research and Development).

Zhou,T. Kim, J., Kalani, A. F., Kim, K., Dunn, W., Jo., Nepomuceno, E., and Liu, F., (2002) In vitro selection of external guide sequences for directing RNase P-Mediated inhibition of viral gene expression, Journal of Biological Chemistry, vol. 277 num. 33, p-112-120.

### DESCRIPTION OF THE INVENTION

In 1956 the inventor sought out to cure sickle cell anemia when it was not heard of in the medical community. The discovery came when viewing a blood cell developmental chart. This was 1970s. When these cells (Stem cells) were found to take on the characteristics of the environment, it was the inventor's ideas that this might help with the GvHD of 1970. Later the inventor realized he had stumbled upon a way to replace all the tiny blood cells in the body. Much research had to happen that the inventor was not allowed to take part in. In the 1980s stem cells were being transplanted into humans and it was met with some success. Although the GvHD and HvGD still proved to be problematic. One of the problems was whether the hemoglobin F gene was being activated from without the cell by a factor since control of Hbg F synthesis could help the condition of Sickle cell. It was found that that there is a factor that regulates the switching of fetal and beta hemoglobin. This factor is a protein which is not the same in not the same in all individuals. Inventor investigated( entire nuclear insertion 1982-1986, and studied transfection from 1973-1982) (Williams, 1990) which would alter the Hbg SS to Hbg AA or Gamma, but it would also alter the HLA. This invention finds a way around this problem by transecting recipient's stem cells with Hbg AA or gamma genes, thereby sparing the HLA type to be perfectly as the original stem cell line. Doctors can decide if the patients need full cell replacement or just globin protein replacement on a case by case basis. Doctors can also decide if they desire multi-potent stem cell transfection or insertion; or progenitor transfection or insertion. In a full cell replacement the HLA type will need to be matched. A method by which Hbg SS can be replaced and the donor HLA remains Intact across the continuum of solid elements of the blood and molecular species such Il-1, GM-CSF, and ICAM.

### SUMMARY OF THE INVENTION

The invention is an in vivo procedure for curing Sickle cell anemia and other disease defined as cellular dyscrasias. The procedure avoids GvHD and HvGD by either altering the defective protein by progenitor transfections for uni-directional differentiation, (i.e. red cells vs. lymphocytes). When the condition calls for change of a protein across the solid elements the transfections will be in the Hematopoietic multi-potent stem cells with renewal capability. The transfections gene therapy is site specific by using cDNA probes, restriction enzymes, transfection or insertion, chaperones, and cell cycling specificity. The transfections are general when the desired gene is introduced in the nuclear domain without site specificity. The transfected cells are cultured and prepared for transplantation. Hematopoietic multi-potent stem cells produce all solid elements, and progenitors produce a specific solid element defined by the specific progenitors. A universal donor is selected for HLA transfections. The recipient's own cells are chosen for other protein gene therapy replacement therapy, such as HbgSS. clinically appropriate amounts of HbgSS will be replaced by HbgAA and or HbgF to increase oxygen saturation by the recipient's red blood cells. Thymus will accept recipient's own lymphocytes thereby preventing GvHD and HvGD.

It is the object of this invention to replace HbgSS in sickle cell anemia patient's blood cells, and or replace a clinically appropriate amount of progenitor cells to replace therapeutic amount of HbgSS laden cells with HLA compatible HbgAA or F producing Hematopoietic progenitor cells.

Sigma factor is the glycoprotein factor that when it binds it's receptors the end stage protein is altered in amino acid sequence hence from HbgF to HbgAA. The switch is at the mRNA level splicing to production of various mRNAs. As many as three Hbgs have been found in a single red blood cell. The hemoglobin producing program switches from producing HbgF to producing HbgAA (Beta-hemoglobin).

The invention consist of a method to replace the beta globin gene in renewable multipotent stem cells at specific sites in the genome to cure sickle cell anemia such that the globin vector only express it self into solid elements from the Hematopoietic progenitors, or any other progenitors for other conditions. Transfection into the recipient's stem cells would avoid the GvHD and HvGD situation. Beta globin gene chaperones lead HbgAA or HbgF to restricted site in genome chromosome 11.

Stem cells are arrested in interphase and nuclei removed, for HbgAA binding and restriction, using HbgAA cDNA probe. HbgAA DNA with valine in the number 6 position of the hemoglobin chain will be removed, and the HbgAA with glutamate in the number 6 position of the hemoglobin chain, or the HbgF will be annealed to the genome and the nucleus reconstituted and replaced back into recipient's stem cell cytoplasm and nuclear envelope.

It is the objective of this invention to restrict the globin gene stem cell interphase. By introducing the Sequence Listing into the stem cell the thymus will deplete those lymphocytes that are not tolerant to the recipient, transplants performed before age 1 or 2 years of age will dimenish an immune attack, and reduce restraint of autologous transplants thereby allowing iso and allo-type transplants, after age 2 only autologous and type O transplants are indicated. Diagnosed the condition, identify missing protein, for each clinical condition. Use triflourinated nucleotides (anti-metabolites) only when live un-attenuated viruses are used in the transfection process. Various viral vectors are available with different capabilities.

### The natural beta hemoglobin sequence is 5′-ε-γg-γa-σ-β-3′.

### DESCRIPTION OF THE BACKGROUND ART

In 1910 James B. Herrick presented an article on a case of anemia with sickle cells.

### In 1943 Medawar and associates discovered the link, or lack thereof, between immature cells and Tolerance.

In the 1950s to cure a genetic blood disease was mire childhood dreams and unheard of. Scientist learns to diagnose sickle cell anemia using cellulose acetate chromatography and citrate agar chromatography. Early embryologist suspected stem cells to be usable to treat human diseases in 1954 but there were no specifics to a cure of any one particular disease or disease type. In 1956 the inventor thought to cure Sickle cell anemia. Stem cells were adopted because stem cell or embryonic cells have the ability to take on the morphological characteristics of the surrounding tissues when transplanted in the same embryo or different species, and they produce all solid elements. Stem cell transplants of the 1980s met with some success however, there still remained the GvHD and HvGD problem. This concept was clarified by the inventor and taken several steps further to replace large numbers of red blood cells while avoiding the HLA problem of GvHD and HvGD. The avoidance of the HLA problem was not tolerated as the immature cells engrafted into the recipient's bone marrow stroma as hoped. This avoidance of the HLA problem had to be overcome by additional means. Viral transfections were discovered by Chase and his associate and other scientist began introducing genes into cells for various purposes, such as to produce hormones. Transfections of progenitor hematological cells left the door open for Hematopoietic stem cell transfections and gene insertion or gene therapy. The ability to replace red blood cells that are defective in a protein or group of proteins, avoid the GvHD and HvGD, plus replace the defective protein such as HbgSS with varying amounts of HbgAA or HbgF has still proven difficult if not impossible. Such an approach has been invented, and is described below.
The ability of stem cells to take on the character of the surrounding is limited by it's inability to return to embryonic stem cell from peripheral stem cells, however stem cells from the periphery can function as Hematopoietic stem cells to produce progenitors of all solid elements in the bone marrow or the embryo. The source of stem cells can be from the embryo, bone marrow, or peripheral blood stream. Any of these stems cells can function in the other environment.

### FIELD OF THE INVENTION

This invention relates specifically to curing Hematopoietic Blood diseases by transplanting clinically useful quantities of perfectly matched stem cells (that differentiate into mature Hematopoietic Pluripotent cells) into autologous host.

In 1956 the inventor sought out to cure sickle cell anemia when it was not heard of in the medical community. The discovery came when viewing a blood cell developmental chart. This was 1970s. When these cells (Stem cells) were found to take on the characteristics of the environment, it was the inventor's ideas that this might help with the GvHD of 1970. Later the inventor realized he had stumbled upon a way to replace all the tiny blood cells in the body. Much research had to happen that the inventor was not allowed to take part in. In the 1980s stem cells were being transplanted into humans and it was met with some success. Although the GvHD and HvGD still proved to be problematic. One of the problems was whether the hemoglobin F gene was being activated from without the cell by a factor since control of Hbg F synthesis could help the condition of Sickle cell. It was found that that there is a factor that regulates the switching of fetal and beta hemoglobin. This factor is a protein which is not the same in not the same in all individuals. Inventor investigated( entire nuclear insertion 1982-1986, and studied transfection from 1973-1982) (Williams, 1990) which would alter the Hbg SS to Hbg AA or Gamma, but it would also alter the HLA. This invention finds a way around this problem by transecting recipient's stem cells with Hbg AA or gamma genes, thereby sparing the HLA type to be perfectly as the original stem cell line. Doctors can decide if the patients need full cell replacement or just globin protein replacement on a case by case basis. Doctors can also decide if they desire multi-potent stem cell transfection or insertion; or progenitor transfection or insertion. In a full cell replacement the HLA type will need to be matched. A method by which Hbg SS can be replaced and the donor HLA remains Intact across the continuum of solid elements of the blood and molecular species such Il-1, GM-CSF, and ICAM.

### DESCRIPTION OF THE FIGURES

FIG. 1.0 Peripheral human blood smear.

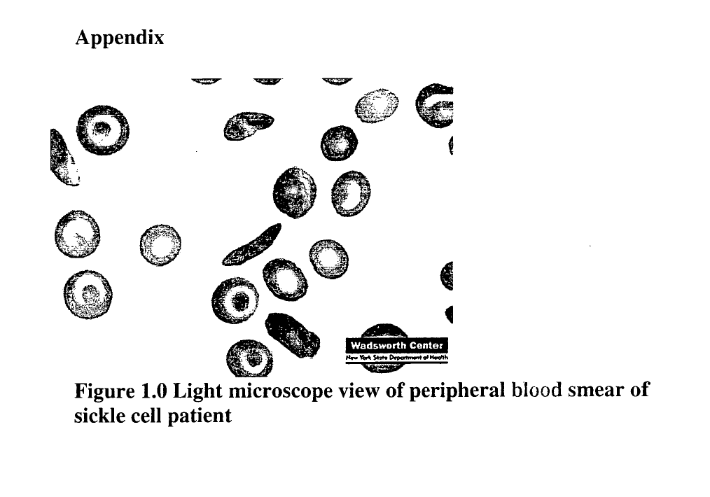

FIG. 2.0 Scanning electron micrograph of sickled and normal red blood cells.

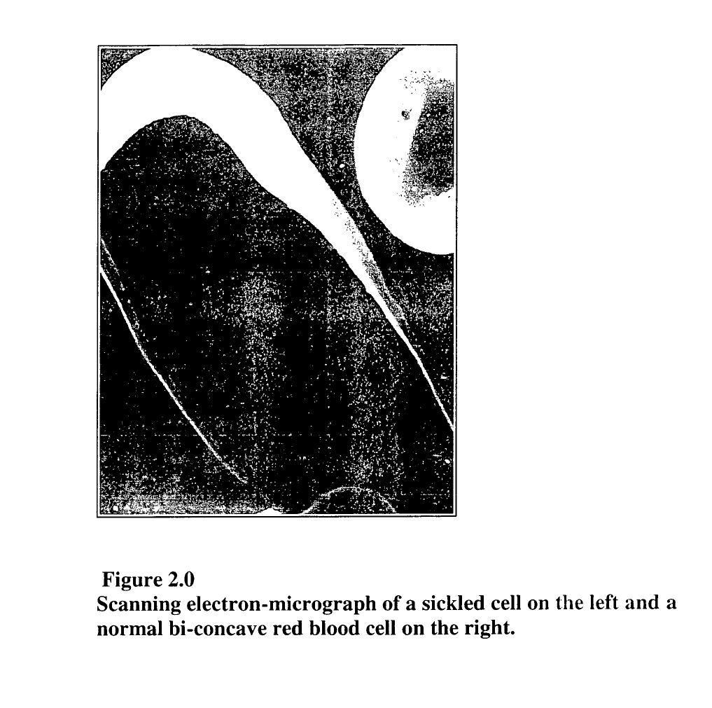

FIG. 3.0 Three dimensional hemoglobin beta amino acid chain.

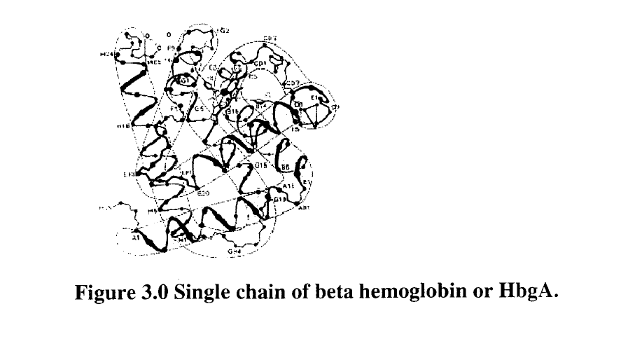

FIG. 4.0 Quaternary structure of HbgAA with two alpha chains, and two beta chains.

FIG. 5.0 Major histo-compatibility and viral transfection scheme.

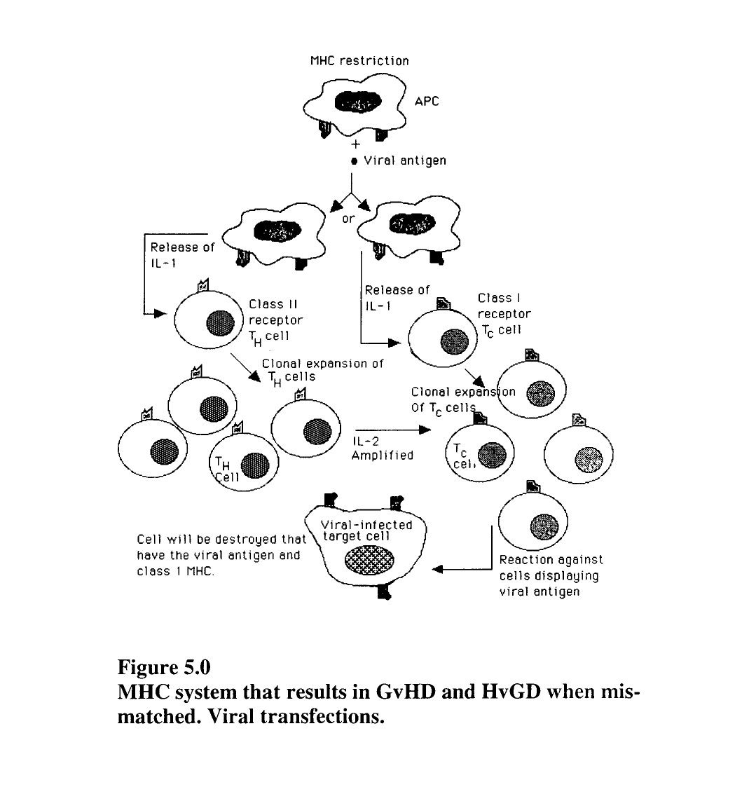

FIG. 6.0 Blood chart showing differentiation of all solid elements from multi-potent stem cells and progenitors.

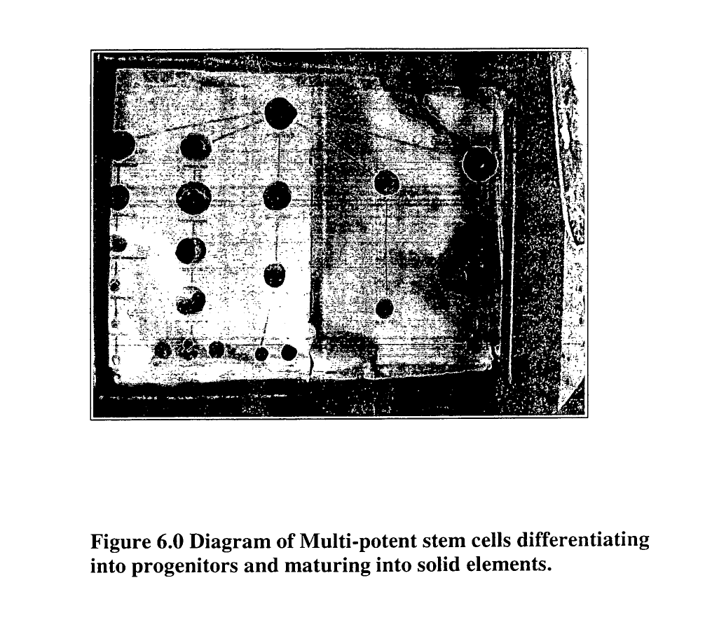

FIG. 7.0 The phases of the cell cycle.

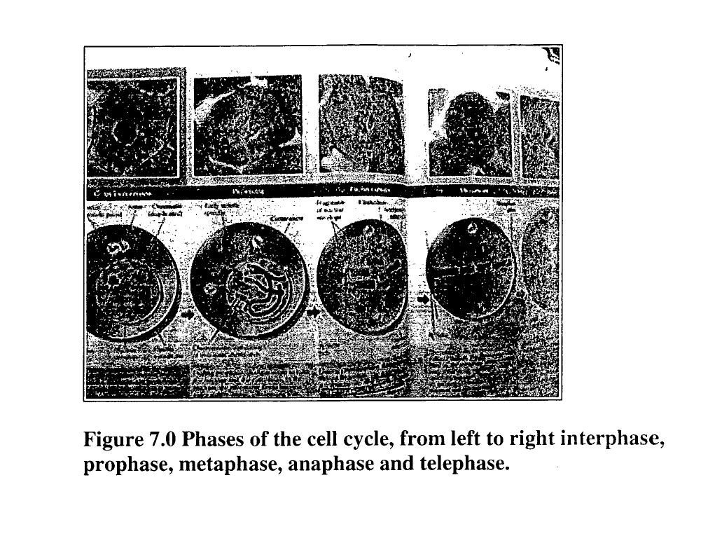

FIG. 8.0 LCR region of beta like globin gene.

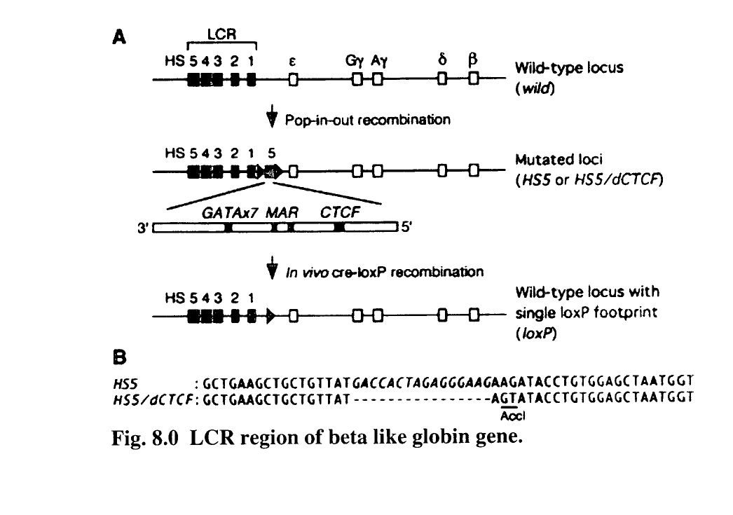

FIG. 9.0 Human stem cell cycle, and arrest for gene transfer.

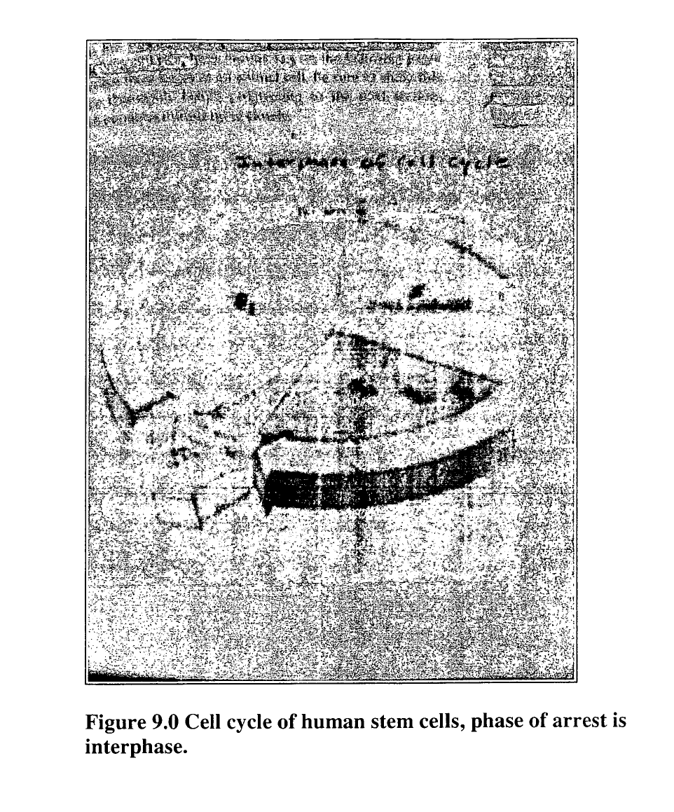

FIG. 10 Hemoglobin Alu gene family sequences.

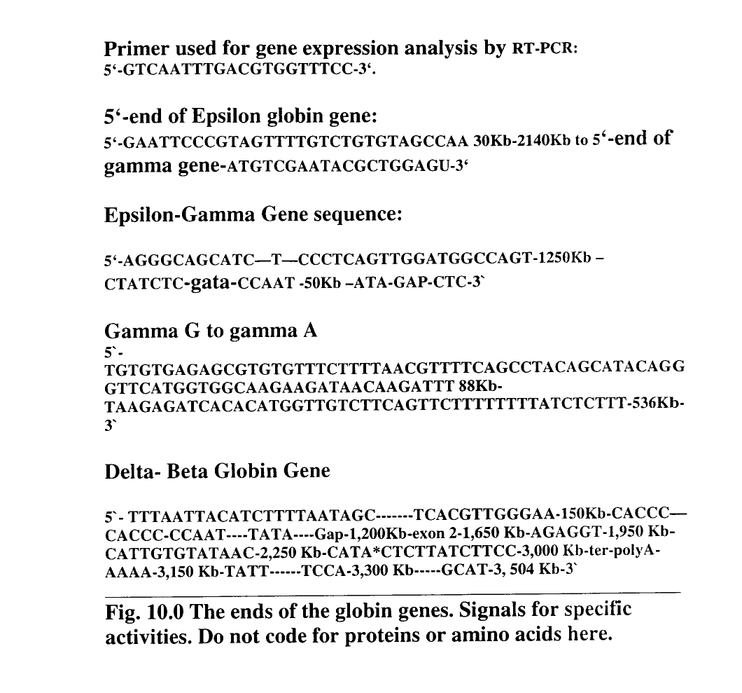

### DETAILED DESCRIPTION OF THE PREFERRED EMBODIMENT

The expression from type O blood is preferred because of a lack of other blood type antigens, for patients with or without functional thymus ( The transplanted cells will replace all solid elements of the blood, of just the element desired) Transplants are for all patients. The multi-potent stem cells will produce all solid elements in physiological amounts. Type O cells will be devoid of other blood group antigens, except where the recipient's cells are used for the transplant. Progenitor cells may be of any of the Hematopoietic series: Colony forming unit-granulocytes, erythrocyte, monocytes and megakaryocyte, eosinophils

The bone marrow produces all solid elements and their progenitors. These cells respond to a number of regulators that promote maturation, and differentiation, protein switching such as colony stimulating factor for each progenitor, stem cell growth factor, and sigma factor.

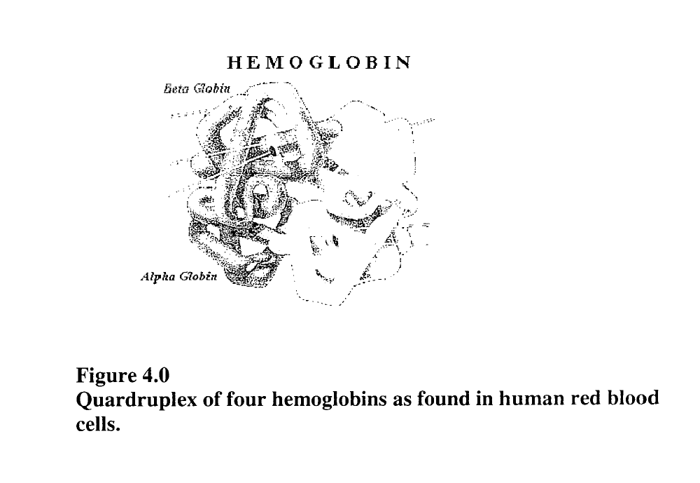

As each protein system is different, it will call for different measures to meet the demands of the particular patient. Hbg BETA has two chains of alpha hemoglobin and two chains of beta hemoglobin. See appendix FIG. 4.0.

The expression of progenitors for all solid elements with specific differentiated end products such as hemoglobin being corrected from HbgSS to HbgAA and or HbgF. This done with or without altering the HLA genes. To produce clinically appropriate amount of red blood cells with HbgAA and or HbgF to increase the oxygen carry capacity of the recipient's red blood cells, there by curing the clinical problem of sickle cell crisis.

For the purpose of this specification and the claims the following definitions and abbreviations will be used:

1. Hemoglobin (Hbg) -oxygen carrying protein of the red blood cell of humans.

2. Fetal Hemoglobin- HbgF, carry oxygen with a greater affinity.

3. Hemoglobin beta (HbgAA)- normal oxygen carrying protein which is defective in sickle cell anemia.

4. Multi-potent stem cell-stem cell capable of expressing all solid element progenitors.

5. Solid elements- erythrocytes or red blood cells (RBC), lymphocyte which cause GvHD and HvGD, neutrophiles which are involved in inflammation, megakaryocyte which are precursors of platelets, monocytes the precursors of macrophages, and basophile.

6. Graph versus host disease- GvHD the recipient of a transplant is not tolerant of the transplant and the transplant began to attack the host or recipient as foreign. Carried out by donor lymphocytes.

7. Host versus graph disease- HvGD the host immune cells are not tolerant with the transplant and the host cells immune cells began to attack the transplant or graph. Carried out by recipient's lymphocytes.

8. Progenitor- Cells derived from multi-potent stem cells that are capable of expressing and producing each of the solid elements specifically as they differentiate.

9. Universal donor cell- used only when HLA has to be altered, formally known as “type O” blood which does not carry lansteiner antigen such as (i.e. A antigens, B-antigens, and therefore does not cause the production of the alternate antibody (A produce anti-B, and B produce anti-A antibodies which are responsible for the formation of antigen antibody complexes that are harmful to the human blood system.

10. Bone marrow- medulary site for all blood solid element formation.

11. Differentiation and switching- process by which immature red cells and other solid elements become the producer of the final type of protein programmed by ontogeny. (e.g. HbgF cells becoming HbgAA cells). The immature cells becomes a mature cell.

12. Cell cycle- the growing cell goes through a cell cycle from Mitosis to interphase and back to mitosis. Interphase consist of Go, G1, S, and G2 phases.

The invention is based on the fact that in order to produce HbgF and or HbgAA in a sickle cell anemia individual continually the multi-potential stem cells must be altered at the beta globin gene such that only the erythropoietic progenitors produce the final product which are red blood cells with multiple Hbgs such as HbgAA and or HbgF. This method does not have to alter the HLA antigens and therefore produce perfectly matched and compatible cells that do not produce GvHD or HvGD. The progenitors under the influence of the recipient's bone marrow environment will regulate the differentiation process.

### Typical Protocols are Described Below Stem Cell Preparation:

As a method that follows the trend, heparinized bone morrow or peripheral stem cells by aspiration from the ileac crest, or venipuncture of the brachial vein of the arm respectively of the recipient or donor. O type blood is preferred where the HLA has to be altered. Accepted surgical and venipuncture procedures are used to collect the above samples of stem cells. One unit or 500 ml of blood is stored at −170 degrees Fahrenheit for later use. Reticulum is removed from the bone marrow by passing the marrow through a micro nylon mesh. The stem of peripheral blood are siphoned from the top of the blood sample after centrifugation.

### Culture Medium

Bone marrow and peripheral stem cells are transferred to a long term medium consisting of AIM-V medium (Gibco, Grand Island, N.Y.) supplemented with insulin (Eli lilly & Co. Indianapolis, Ind.) at 10 ml mu. g/ml, human albumin (American Red Cross, Washington, D.C.) 50 mg/ml, saturated human ferritin (Sigma Co., St Louis, Mo.) at 200 mu.g/ml, hydrocortisone (sodium succinate) (Upjohn Co. Kalamazoo, Mich.) at 10 sup.-6 M, cholesterol (C3045, Sigma Chemical Co.) at 7.5 mu. g/ml, and liposyn II (10% Abbot Labs., No. Chicago, Ill.) at 0.05 l.ml medium. In addition, penicillin G potassium (Roerig div of Pfizer, Inc. New York, N.Y.), gentamicin sulfate (Schering Corp., Kenilworth, N.J.) and amphotricin B (Bristol Myers-Squibb, Princeton, N.J.) are added to the stem cells as preservatives. Iscove's modified Dulbecco, Fisher's or Eagle's media are used. In addition, fetal calf serum or horse serum may be substituted for human serum.

### Arresting Cells in Interphase

The stem cells are arrested at interphase by decreasing the oxygen concentration after Van Pelt (2005). Treat stem cells with Ara-C (100 mg/kg), using 7-aminoactinmycin-D (7-AAD) for DNA staining. Treat with bromodeoxyuridine, 2 hrs later cells cease to incorporate BrU, after 4 hrs s-phase arrested cells began to activate. 28% of cell will incorporate BrU at 20 hrs. After 72 hrs the cells recover from the arrest, returning cells to Go phase.

### Transfection:

Removal of the interphase nucleus, excision of the defective globin gene and replacement of beta globin gene with HbGAA or HbgF, annealing of excised DNA and replacement of nucleus. Cells are infected with the virus-A phophoglycerate kinase promoter driven expression of a green fluorescence protein (GFP) cDNA, and an anti-sickle HbgAA or HbgF globin genes under the control of HS2, HS3, HS4 enhancers.

### Bioreactors

Transfection of stem cells and isolate them to be tested for desired progenitor incorporating the HbgAA or HbgF genes. And Bioreactor culture systems, to mutiply the stem cells. Opticell. T.M. Optocore.TM. ceramic core S-51, S451 (flat surface area 23.8.sup.2), S-1251 (flat surface area 10.4m.sup.2) or S-7251 (Cellex Biosciences, In., Minneapolis, Minn.) are preferred. These bioreactors are initially sterile perfused, preferably for 1-3 days, with sterile deionized water to remove any toxic substances adhering to the core. Therefore, the core is perfused for a brief period (less than 24 hrs.) with sterile 25% (w/v) human serum albumin in order to coat the core with protein. The bioreactor core is next perfused for 4-24 hrs with a sterile solution of an anticoagulant, preferably heparin sulfate, 100 U/mL (Upjohn Co.) as a source of glycosaminoglycan and to prevent cell clumping during stem cell inoculation. Following this preparation, the core is conditioned by perfusing it with sterile human stem cell medium (see culture mediums above), preferably for about 24 hours, prior to inoculating the bioreactor with stem cells. The stem cells that produce the altered Hbg will be cultured in a bioreactor (as above) to clinically useful number of stem cells for transplant. For procedures see (Oh, 2004). Self-renewal cells take up the viral transfected beta globin gene.

### Bioreactor Culture System

The culture system consist of a variable number of bioreactors connected to the medium source by sterile plastic tubing. The medium is circulated through the bioreactor with the aid of a roller or centrifugal pump (e.g., KOBE>TM). Probes to measure pH, temperature, and O.sub.2 tension are located in line at points immediately before and following the bioreactor(s). Information from these sensors is monitored electronically. In addition, provision is made for obtaining serial samples of the growth medium in order to monitor glucose, electrolytes, Growth factors, and other nutrient concentrations.

### Inoculation of Bioreactor with Altered Pluripotent

### Hematopoietic Stem Cells

Multiplying, and Altered Pluripotent Hematopoietic Stem cells

Appropriate for the size of the bioreactor, at a concentration of about 2 times. 10sup.7 cells/ml., are mixed with an equal volume of autologous fresh stem cells are injected into the bioreactor. Circulation of growth medium is interrupted for a period of about 1-4 hours such that the cells are allowed to attach to the core of the bioreactor core capillaries. Thereafter, the circulator pump is engaged and the growth medium pumped through the system at an initial rate determined by the size of the reactor, atypical rate is about 24 ml/min. Gas exchange occurs via silicone tubes (surface area=0.5 m.sup.2) within a stainedless steel shell, or by a conventional membrane oxygenator. O.sub.2 and pH are monitored continuously by polarographic O.sub.2 probes and autoclavable pH electrodes, respectively. Flow rates are adjusted so as to maintain an optimal O.sub.2 tension (a partial pressure of at least about 30-50 mm of Hg) and optimal pH (7.30-7.45).

When an appropriate number of vector carrying stem cells have been obtained (approximately 5-10 mililiters), as determined by oxygen utilization of the system, a second bioreactor may be connected to the system, and cells fed directly into this second bioreactor. Thereafter, the second bioreactor is flushed with growth medium containing a high concentration (e.g. 10,000 U/mL) of EPO or other differentiation factors, and maintained for 1-3 days for final maturation of the desired blood components, (i.e. multi-potent-potent stem cells).

### Cell Harvest and Processing

The bioreactor(s) is (are) mated with a conventional cell separator, and the cells are collected from the core or capillaries with gentle agitation. Harvested blood cells are processed in an automated cell separator and placed in sterile blood bags for transfusion.

Bags of stem cells may be irradiated conventionally and tested for any contamination during refrigeration for 1-3 days. Neomycin resistant gene as a selection process, after (Mansour, 1988). Efficiency may be as high as 85%.

Thus, the invention can also provide a single multipotent stem cell line species for the cure of various previously uncurable diseases. This achieved by expanding the culture of multi-potent Hbg globin altered self-renewing Hematopoietic stem cells in the bioreactor until cell numbers are clinically appropriate to re-introduce them back into the patient or the recipient.

What is claimed:

1. technology to use stem cells to Cure of Sickle Cell Anemia by perfect match (Autologous transplants), a new source of blood, A method for isolation of Hematopoietic Pluripotent stem cells (The recipient) of HbgSS individual CD34 variable capability and perfect matching of HLA typing, And clinical decision to perform a whole cell transplant or transplant of only the hemoglobin transfected Hematopoietic stem cells. Cure for GvHD and HvGD.
There is no need for as much chemotherapy or radiation therapy, with antibiotic. Selection of pure stem cell line with no active infections. Defined as no viral cell cycle evidence in cell line. Cells may be treated with thymindine nucleotide derivatives to stop viral cycling. The Transformed cell line. The self renewal of Hematopoietic stem cells vs. the non-renewal of Hematopoietic progenitor cells after transfection or electroporation. The ability of the stem cell to be transplanted from any source (i.e. embryo, bone marrow, or peripheral blood) to any of the other locations (e.g. bone marrow) after transfection or electroporation. The transfection of stem cells to alter proteins as gene therapy for genes that are defective and produce non-functional proteins. The gene sequence can be found on the accompanying CDs: Cure for Sickle Cell Disease, Bruce M. Williams, Beta globin gene, created Sep. 19, 2006.

2. Method for culturing, and transplanting recipient compatible stem cells into patient suffering from Sickle cell disease (See results in Williams, Mar. 28, 1990) Sickle celled anemia and other blood dyscrasias, including leukemia's. Method can be used for any condition or disease where a protein needs to be replaced. In the event of anemias without protein abnormality within the Hematopoietic system stem cells can be multiplied and transplanted to correct the anemia. Transplant is in a distributed pattern as islets only have one stem cell surrounded progenitors and partially differentiated cells throughout the bone marrow. Wherein red blood cells can contain multiple hemoglobins, such as Hbg AA, Hbg F, and Hbg Beta can exist in the same cell simultaneously after transfection or electroporation.
Gene therapy for HLA—not Hemoglobin (Hbg) SS only. May still need HLA matching with stem cell transplant for HLA A, B, and DR (Bone, 1992). Non-identical allograph mis-match at some MHC loci. The recipient's loci for the mismatching loci can be inserted via needle or transfection (Barrett, 2003), or electroporation. A mixture of antigen has a better outcome clinically (Fernandez, 2003). Low probability of cancer and possibility for a restored immunity or newly acquired immunity.
Allotypes, and sibling matches for whole cell replacement. Replacing billions of microscopic cells naturally, with vector (HbgF and or HbgAA) being expressed coordinately in the specified Hematopoietic cell line continuously without the need for repeat transplant procedures performed on the recipients. The growth potential, immortality, gene manipulation of transfected or electroporated stem cells. Transposition of globin gene elements after transfection or electroporation.
Increasing the chance of finding a compatible match that donor is tolerant to or no need for compatible match.
Thymus instructed by this method to select tolerant lymphocytes for donor by inserted recipient's HLA gene mismatched in donor cells after transplant, transfection, or electroporation.
Hemoglobin switching factor transfection or insertion.
Developing cells modify to the donor environment, mature lymphocytes are not present and tolerant cells are selected by the thymus of the donor. The transplanted cells remain the appropriate cell type for the environment after transfection and in-vivo manipulations of stem cells for cell culture techniques, as well as the ability to continue self-renewal.
Moderation of GvHD and HvGD. Reactive lymphocytes do not have to be separated from the tranplantsate before transplant procedures are performed.

3. Stem cell gene insertion method for CD, HLA, and HbgAA. Continual renewal of transplanted HLA transfected Stem cells in vivo, and hence continual production of HbgAA Donor HLA compatible Hematopoietic cells. 5′ to 3′ end of Globin gene. 5′-epsilon-gamma-delta-beta-3′ must be the end result of the transplantation regardless of the section transfected. Only the beta gene can be transfected but all of the necessary component of the gene must be present in a mitotically active cell because recombination occurs with in the globin gene cluster or complex, which includes promoter, operator, Alu sequences, Knpl Sequences, and repetitive sequences, and regulatory sequences. Methylation will occur at the gene that should not be active at the stage of ontogeny the transplant is performed. Regulatory insertions can increase the production of fetal hemoglobin or beta hemoglobin if desired. Single stranded cDNA is recombined to duplex DNA before transfection. After transfection or electroporation, and cell cycling the enzymatic insertion of the transfected or electroporated globin gene into the complete globin gene site. Transfection or electroporation during prophase or telephase to avoid methylation and to assure incorporation of the corrected gene into gene site to produce mRNA for the various hemoglobins during development. The earlier the procedure is performed the better the outcome in ontogeny.
Minor HLA factor can also be transformed, transfected (complex-globin genes HbgAA and HbgF or inserted with a matching vector, best practice-Decision whether to transfect HLA or not (Shinar, 1989). Transforming Hbg SS stem cells into Hbg AA synthesizing cells after transfection or electroporation. Maintenance of (the HbgAA and HbgF genes are linked in tandem) and variably expressed, or co-expressed during development and differentiation after transfection or electroporation.
Incubating transformed HbgSS stem cells in a bioreactor in a growth medium recombinant human growth hormone, or maturating promotion polypeptides, to differentiate in vivo or vitro into singular derivatives or multiple derivatives (i.e. erythrocytes only or erythrocytes and the other solid elements such as granulocytes, and lymphocytes).
Limit the number of transfections as much as Possible.
Improvements in the transfections procedure.
Harvesting said HbgAA transfected or electroporation stem cells after gene insertion from cultures in agar, HEBES, MEM, and Eagle media.
Chosen Stem cells are generally free of active viral cycles. Decisions can be made as to whether to interrupt a viral cycle in a given case.
Reduced chance of cancer.
Human Genome -presence of viral genes in human stem cell genome. The resultant stem cell genome after transfection or electroporation, and substituted thymidine treatment of stem cells.
The resultant Hematopoietic cells do not have to have 100% Hbg AA blood. A clinical decision can be made as to what percentage of HbgAA genes should be transfected, keeping in mind to transfect as little as possible.
The method of claim 1 in which an exogenous protein is expressed in donor cells and functions in recipient's or donor cells, which ever is decided to be transformed by cases by case basis. From research any where from ¼ to ½ of the Hbg can be represented as HbgF, or Hbg AA.
The method of claim 1-19, (Best practice decision or alternative to be made) in which nucleus of hematopoietic stem cell is removed and cDNA of HbgSS gene is identified on chromosome 11, or any chromosome for other conditions, and excised using restriction enzymes, (Competition) the HbgF and or HbgAA can be introduced and the nucleus returned to the stem cell (Melton, (2004). This does not necessarily have to be done

4. The method of claim 1 whereby (Also donor in some cases) recipient HLA perfectly matched stem cells differentiates into recipient's HLA compatible HbgF and or HbgAA producing Erythroid series, granulocyte series, megakaryocyte series and the lymphocyte series.

5. The method of claim 1 Recipient HLA Compatible monocytes and macrophage series.

6. The method of claim 19 whereby all immune reactive species are Donor HLA specific, and tissue specific including but not limited to SCGF, Il-1, Il-3 and ICAM and VCAM genes for adhesion in bone marrow and cultures, signal transduction and down regulation of HbgSS gene.

7. The method of claim 14 whereby the best practice is used to determine the most efficient and safe way to introduce the HbgAA or HbgF genes into the cell cycle phases, such as during the DNA synthesis phase and the mitotic phases of interphase and telephase, to avoid DNA methylation and DNA degradation enzymes from destroying the introduced gene(s), stopping the vector from duplicating, distributing, and transcribing.

8. The method of claim 3 whereby restriction of sickle globin gene by restriction enzymes, and ligation of HbgAA-HbgF genes into chromosome 11 genetic material. Restriction Enzymes- Decision to either use cell nuclei removal or stem cell transfection or electroporation only, to replace defective gene(s) (i.e. HbgSS). Selection of restriction enzymes. Removal of viral genes in-vitro before transfection from the genomic (i.e. globin, insulin) genes.
Improvement of the hemoglobin oxygen saturation curve in Hbg SS positive patients, and reduction of the sickle crisis after transplant procedures is administered.
Follow-up to test success of transplant by measuring hematological parameters such as % Hbg AA and or Hbg F in the recipient's peripheral blood.
The method of claim 1 whereby Other conditions that might benefit from this technology are osteoporosis to replace osteoclast progenitors, and diabetes where beta islet cell progenitors can be transformed to produce insulin synthesizing beta islet cells and remain compatible. Many other conditions and disease can be ameliorated or cured by this technique (i.e. leukemia, thalassemia). It suffices to claim that any tissue diseased in the entire body can be replaced in this way via (i.e. mesemchyme of neuroblast, astroblast, and myoblast). Extension of disease treatment- Use of stem cells as individual's model for drug testing on humans and future research. A replacement for white mice.


SEQUENCE LISTING (1 sequence)

SEQ ID NO: 1 - DNA, 81706 bp, homo sapien
ggatcctcacatgagttcagtatataattgtaacagaataaaaaatcaattatgtattcaagttgctagtgtcttaagaggttcacatttttatctaactgattatcacaaaaatacttcgagttacttttcattataattcctgactacacatgaagagactgacacgtaggtgccttacttaggtaggttaagtaatttatccaaaaccacacaatgtagaacctaagctgattcggccatagaaacacaatatgtggtataaatgagacagagggatttctctccttcctatgctgtcagatgaatactgagatagaatatttagttcatctatcacacattaaacgggactttacatttctgtctgttgaagatttgggtgtggggataactcaaggtatcatatccaagggatggatgaaggcaggtgactctaacagaaagggaaaggatgttggcaaggctatgttcatgaaagtatatgtaaaatccacattaagcttctttctgcatgcattggcaatgtttatgaataatgtgtatgtaaaagtgtgctgtatattcaaaagtgtttcatgtgcctaggggtgtcaaatactttgagtttgtaagtatatacttctctgtaatgtgtctgaatatctctatttacttgattctcaataagtaggtatcatagtgaacatctgacaaatgtttgaggaacaatttagtgtttacctattcaccaaaatttattaaatgcctaatctgtatcagatatacaattatctggcgaaatctgtaattcctaatttaaacagctgtgtagcctaattagggataaaggcatgcaaacccataatttgtgtaggttgaaatgagctatagaaaaatgcagtatatttatcagaagtctttagggtcatgaaaaggaatggtcaactgacactgccagggactcatatgtaagagataactaatgtgaagtgactttaaaggagaaattagcagaagttttctttccatgtctcctcatcatgttacaataacggaagagattaaaacaacaaatacatttagacagcaatgtttatcctggttagatgttttaatctaaatctatcttggagtgttaaaatgcatttgctcacctactttaaaatataaatgaaggtaggaacctgtagatacaaaaagttggagaaaaaaagacaataaagatgacaaaaatctattaatccttgatagaaaatgagaagagataaaacactggtttacataaagaaaataagatggatagatagcagatccttataaaagtgataatttgagaaaaaaaatactccatattctgagtttcttcacataaaataatacaaatctgctgtggtaagttacaaagagatagattttttatcattatataaaagatattttaaacagagttatacaacaaaggaacagactatgtcatatattctcacttatcactataaacatctcagaaaaatctgcaaaatcatttcatagcattttaaatagttaggaataatgtagaaaactgaaacagttctaagtttcccacaaacttagagtctcaaatgttgcattacctaacttacctgcaaatattttatacaaatttgcacatgctactctagtcaaaaatatatgtacattatgggtattttctgtgtgtaacttggttctagttgcttctttcagaaatagcctctatttttgatttacctgataaaatcacattcctctccaaagccttctaaatacttccagactaactactttttagtacatctaagaagaaaagagttttgtctcttatccacctctgagtcaaaaagcagcatgtccatcaattggtacatagttcccacagccccacttagctctggattggagttctacttggcattgtttgcaactacatggacgtaaaatgcatggattctcttgaaaaaatgtttctgccatgatgttctctgaaagagactaaccttccctcgctttgcagagaaagactcgtgtaatccttgacaatgtcatctcatctatttattcccatgtctacccatatgtgaccttcatgtctttgctctaagcccctacatcctcaatctacacactaggatagtataaaagtaatagtaataatagtagtaatagtaataacaatacaatgattatggcttatactatacacaagacactgttgatatattatttcatttagtattcacagtaactctgtgcctcaagtactattgtaataccctttaagaggaggaaactgaggcacagggccctaaagtaatattccaagatgaagtggctactaactgacagagggcataattcaactcatgatatttggctctagaatacatgctctgaatcattatacaataataattcatgaggaaacattttttaaagcctaagttatttgctctgaaataagacataatttggggtgagaaagcttagattccatgaagtattacagcatttggtagtctttttgcactccaggtcttatttttactgcttaaacataataaaacatatggttcagtatgcctttgattttacaataatattcctgttatttttggaagcacagggtgtgggataatgctaattactagtgattagtattgagaggtgacagcgtgctggcagtcctcacagccctcgctcgctcttggcgcctcctctgcctgggctcccacattggtggcacttgaggagcccttcagccggccgctgcactgtgggagcccttttctgggctggccaaggccagagccggctccctcagcttgccaggaggtgtggagggacagacgcgggcaggaaccgggctgtgcgccgtgcttgagggagttccgggtgggcatgggctccgaggaccccgcactcggagccgccagccggccccaccggccgcgggcagtgaggggcttagcacctgggccagcagctgctgtgctcaattcctcgccgggccttagctgccttcctgcggggcagggctcgggacctgcagcgcgccatgcctgagcctccccaccttcatgggctcctgtgcggcccgagcctcgccgacgagcgccgccccctgctccagggcacccagtcccatcgaccacccaagggctgaagagtgcgggcgcacggcaggggactggcaggcagctccccctgcagcccaggtgcgggatccactgggtgaagccggctaggctcctgagtttgctggggatgcgaagaacccttatgtctagataagggattgtaaatacaccaattggcactctgtatctagctcaaggtttgtaaacacaccaatcagcaccctgtgtctagctcagggtttgtgaatgcaccaatcaacactctatctagctactctggtggggccttggagaacctttatgtctagctcagggattgtaaatacaccaatcggcagtctgtatctagctcaaggtttgtaaacacaccaatcagcaccctgtgtctagctcagggtttgtgaatgcaccaatcaacactctgtatctagctactctggtggggacgtggagaacctttatgtctagctcagggattgtaaatacaccactcggcagtctgtatctagctcaaggtttgtaaacacaccaatcagcaccctgtgtctagctcagggtttgtgaatgcaccaatcaacactctgtatctagctactctggtggggacttggagaacctttgtgtggacactctgtatctagctaatctggtggggacgtggagaacctttgtgtctagctcatggattgtaaatgcaccaatcagtgccctgtcaaaacagaccactgggctctaccaatcagcaggatgtgggtggggccagataagagaataaaagcaggctgcccgagccagcagtggcaacccgctcgggtccccttccacactgtggaagctttgttctttcgctctttgcaataaatcttgctgctgctcactgtttgggtctacactgcctttatgagctgtaacgctcaccgcgaaggtctgcagcttcactcttgaagccagcgagaccacgaacccaccgggaggaacgaacaactccagaggcgccgccttaagagctggaacgttcactgtgaaggtctgcagcttcactcctgagccagcgagaccacgaacccatcagaaggaagaaactccgaacacatccaaacatcagaacgaacaaactccacacacgcagcctttaagaactgtaacactcaccacgagggtccccggcttcattcttgaagtcagtgaaaccaagaacccaccaattccggacacagtatgtcagaaacaatatgagtcactaaatcaatatacttctcaacaatttccaacagcccttgcaattaacttggccatgtgactggttgtgactaaaataatgtggagataataatgtgttactccctaaggcagagtgcccttctatcattctctttcccttcctctatgtggcagaaagtaaaagattctgaaatgataaagtcaatcacaggaaggcacctggactcctggcccactgcttggaggagagcactcaggaccatgaacatctgactgtgacgtagcaataaagaaacccacgtttcatatgaaactgcttaaaattaatggcacaagtcatgtttttgatgttgcacatttgtctttatttgtggcttgttttgcttccacatcaatccactcaaggcctacattctgctataatgcaatttcaagttctttacaggccgagaaaaatgaatctgaattcctgacctccaaaagtgatcaagatatttttagttcaggctccaaaattttctcattttcataggttttcctcgattgatcattattcatgatttgcaaggaatcattcaatgttttctaaatctattactgcatcctgacacatatgacattttaactatgttccagatttttgaatgaagagtgtaaattttaaatgttttcaccacaaaaaataagtatgtgaagtggtggatttgttaattagccttatttaaccatttaatattgtacacgtacaccaaagcatcatgttgtaccccatgaatacacacaattattatttgtcaatttaaaatgaaataataaaaaataacaaaggcattagcctctgcattgcctttaccggtcatcctcacggtgactaacgcaaaaaacgttctatttcatccttacaaacatccctatctttgatgcctctttgtctagatctctatcccctcctgttttctctacgttatttatatgggtatcatcaccatcctggacaacatcaggacagatatccctcaccaagccaatgttcctctctatgttggctcaaatgtccttgaactttcctttcaccaccctttccacagtcaaaaggatattgtagtttaatgcctcagagttcagcttttaagcttctgacaaattattcttcctctttaggttctcctttatggaatcttctgtactgatggccatgtcctttaactactatgtagatatctgctactacctgtattatgcctctacctttattagcagagttatctgtactgttggcatgacaatcatttgttaatatgacttgcctttcctttttctgctattcttgatcaaatggctcctctttcttgctcctctcatttctcctgccttcacttggacgtgcttcacgtagtctgtgcttatgactggattaaaaattgatatggacttatcctaatgttgttcgtcataatatgggttttatggtccattattatttcctatgcattgatctggagaaggcttcaatccttttactctttgtggaaaatatctgtaaaccttctggttcactctgctatagcaatttcagtttaggctagtaagcatgaggatgcctccttctctgatttttcccacagtctgttggtcacagaataacctgagtgattactgatgaaagagtgagaatgttattgatagtcacaatgacaaaaaacaaacaactacagtcaaaatgtttctctttttattagtggattatatttcctgacctatatctggcaggactctttagagaggtagctgaagctgctgttatgaccactagagggaagaagatacctgtggagctaatggtccaagatggtggagccccaagcaaggaagttgttaaggagcccttttgattgaaggtgggtgcccccaccttacagggacaggacatctggatactcctcccagtttctccagtttccctttttcctaatatatctcctgataaaatgtctatactcacttccccatttctaataataaagcaaaggctagttagtaagacatcaccttgcattttgaaaatgccatagactttcaaaattatttcatacatcggtctttctttatttcaagagtccagaaatggcaacattacctttgattcaatgtaatggaaagagctctttcaagagacagagaaaagaataatttaatttctttccccacacctccttccctgtctcttaccctatcttccttccttctaccctccccatttctctctctcatttctcagaagtatattttgaaaggattcatagcagacagctaaggctggttttttctaagtgaagaagtgatattgagaaggtagggttgcatgagccctttcagttttttagtttatatacatctgtattgttagaatgttttataatataaataaaattatttctcagttatatactagctatgtaacctgtggatatttccttaagtattacaagctatacttaactcacttggaaaactcaaataaatacctgcttcatagttattaataaggattaagtgagataatgcccataagattcctattaataacagataaatacatacacacacacacacattgaaaggattcttactttgtgctaggaactataataagttcattgatgcattatatcattaagttctaatttcaacactagaaggcaggtattatctaaatttcatactggatacctccaaactcataaagataattaaattgccttttgtcatatatttattcaaaagggtaaactcaaactatggcttgtctaattttatatatcaccctactgaacatgaccctattgtgatattttataaaattattctcaagttattatgaggatgttgaaagacagagaggatggggtgctatgccccaaatcagcctcacaattaagctaagcagctaagagtcttgcagggtagtgtagggaccacagggttaagggggcagtagaattatactcccactttagtttcatttcaaacaatccatacacacacagccctgagcacttacaaattatactacgctctatactttttgtttaaatgtataaataagtggatgaaagaatagatagatagatagacagatagatgatagatagaataaatgcttgccttcatagctgtctccctaccttgttcaaaatgttcctgtccagaccaaagtaccttgccttcacttaagtaatcaattcctaggttatattctgatgtcaaaggaagtcaaaagatgtgaaaaacaatttctgacccacaactcatgctttgtagatgactagatcaaaaaatttcagccatatcttaacagtgagtgaacaggaaatctcctcttttccctacatctgagatcccagcttctaagaccttcaattctcactcttgatgcaacagaccttggaagcatacaggagagctgaacttggtcaacaaaggagaaaagtttgttggcctccaaaggcacagctcaaacttttcaagccttctctaatcttaaaggtaaacaagggtctcatttctttgagaacttcagggaaaatagacaaggacttgcctggtgcttttggtaggggagcttgcactttccccctttctggaggaaatatttatccccaggtagttccctttttgcaccagtggttctttgaagagacttccacctgggaacagttaaacagcaactacagggccttgaactgcacactttcagtccggtcctcacagttgaaaagacctaagcttgtgcctgatttaagcctttttggtcataaaacattgaattctaatctccctctcaaccctacagtcacccatttggtatattaaagatgtgttgtctactgtctagtatccctcaagtagtgtcaggaattagtcatttaaatagtctgcaagccaggagtggtggctcatgtctgtaattccagcacttgagaggtagaagtgggaggactgcttgagctcaagagtttgatattatcctggacaacatagcaagacctcgtctctacttaaaaaaaaaaaaaaaattagccaggcatgtgatgtacacctgtagtcccagctactcaggaggccgaaatgggaggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagttcacgtgtgtaaaaaggaggatgcttctttcctttgtattctcacatacctttaggaaagaacttagcacccttcccacacagccatcccaataactcatttcagtgactcaacccttgactttataaaagtcttgggcagtatagagcagagattaagagtacagatgctggagccagaccacctgagtgattagtgactcagtttctcttagtagttgtatgactcagtttcttcatctgtaaaatggagggttttttaattagtttgtttttgagaaagggtctcactctgtcacccaaatgggagtgtagtggcaaaatctcggctcactgcaacttgcacttcccaggctcaagcggtcctcccacctcaacatcctgagtagctggaaccacaggtacacaccaccatacctcgctaattttttgtatttttggtagagatggggtttcacatgttacacaggatggtctcagactccggagctcaagcaatctgcccacctcagccttccaaagtgctgggattataagcatgattacaggagttttaacaggctcataagattgttctgcagcccgagtgagttaatacatgcaaagagtttaaagcagtgacttataaatgctaactactctagaaatgtttgctagtattttttgtttaactgcaatcattcttgctgcaggtgaaaactagtgttctgtactttatgcccattcatctttaactgtaataataaaaataactgacatttattgaaggctatcagagactgtaattagtgctttgcataattaatcatatttaatactcttggattctttcaggtagatactattattatccccattttactacagttaaaaaaactacctctcaacttgctcaagcatacactctcacacacacaaacataaactactagcaaatagtagaattgagatttggtcctaattatgtctttgctcactatccaataaatatttattgacatgtacttcttggcagtctgtatgctggatgctggggatacaaagatgtttaaatttaagctccagtctctgcttccaaaggcctcccaggccaagttatccattcagaaagcattttttactctttgcattccactgtttttcctaagtgactaaaaaattacactttattcgtctgtgtcctgctctgggatgatagtctgactttcctaacctgagcctaacatccctgacatcaggaaagactacaccatgtggagaaggggtggtggttttgattgctgctgtcttcagttagatggttaactttgtgaagttgaaaactgtggctctctggttgactgttagagttctggcacttgtcactatgcctattatttaacaaatgcatgaatgcttcagaatatgggaatattatcttctggaatagggaatcaagttatattatgtaacccaggattagaagattcttctgtgtgtaagaatttcataaacattaagctgtctagcaaaagcaagggcttggaaaatctgtgagctcctcaccatatagaaagcttttaacccatcattgaataaatccctataggggatttctaccctgagcaaaaggctggtcttgattaattcccaaactcatatagctctgagaaagtctatgctgttaacgttttcttgtctgctaccccatcatatgcacaacaataaatgcaggcctaggcatgactgaaggctctctcataattcttggttgcatgaatcagattatcaacagaaatgttgagacaaactatggggaagcagggtatgaaagagctctgaatgaaatggaaaccgcaatgcttcctgcccattcagggctccagcatgtagaaatctggggctttgtgaagactggcttaaaatcagaagccccattggataagagtagggaagaacctagagcctacgctgagcaggtttccttcatgtgacagggagcctcctgccccgaacttccagggatcctctcttaagtgtttcctgctggaatctcctcacttctatctggaaatggtttctccacagtccagcccctggctagttgaaagagttacccatgcagaggccctcctagcatccagagactagtgcttagattcctactttcagcgttggacaacctggatccacttgcccagtgttcttccttagttcctaccttcgaccttgatcctcctttatcttcctgaaccctgctgagatgatctatgtggggagaatggcttctttgagaaacatcttcttcgttagtggcctgcccctcattcccactttaatatccagaatcactataagaagaatataataagaggaataactcttattataggtaagggaaaattaagaggcatacgtgatgggatgagtaagagaggagagggaaggattaatggacgataaaatctactactatttgttgagaccttttatagtctaatcaattttgctattgttttccatcctcacgctaactccataaaaaaacactattattatctttattttgccatgacaagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagctgctgcagtcaaagtcgaatgcagctggttagactttttttaatgaaagcttagctttcattaaagattaagctcctaagcagggcacagatgaaattgtctaacagcaactttgccatctaaaaaaatctgacttcactggaaacatggaagcccaaggttctgaacatgagaaatttttaggaatctgcacaggagttgagagggaaacaagatggtgaagggactagaaaccacatgagagacacgaggaaatagtgtagatttaggctggaggtaaatgaaagagaagtgggaattaatacttactgaaatctttctatatgtcaggtgccattttatgatatttaataatctcattacatatggtaattctgtgagatatgtattattgaacatactataattaatactaatgataagtaacacctcttgagtacttagtatatgctagaatcaaatttaagtttatcatatgaggccgggcacggtggctcatatatgggattacatgcctgtaatcccagcactttgggaggccaaggcaattggatcacctgaggtcaggagttccagaccagcctggccaacatggtgaaaccccttctctactaaaaaatacaaaaaatcagccaggtgtggtggcacgcgtctataatcccagctactcaggaggctgaggcaggagaatcacttgaacccaggaggtggaggttgcagtgagctaagattgcaccactgcactccagcctaggcgacagagtgagactccatctcaaaaaaaaaaaaagaagtttattatatgaattaacttagttttactcacaccaatactcagaagtagattattacctcatttattgatgaggagcccaatgtacttgtagtgtagatcaacttattgaaagcacaagctaataagtagacaattagtaattagaagtcagatggtctgagctctcctactgtctacattacatgagctcttattaactggggactcgaaaatcaaagacatgaaataatttgtccaagcttacagaaccaccaagtagtaaggctaggatgtagacccagttctgctacctctgaagacagtgttttttccacagcaaaacacaaactcagatattgtggatgcgagaaattagaagtagatattcctgccctgtggcccttgcttcttacttttacttcttgtcgattggaagttgtggtccaagccacagttgcagaccatacttcctcaaccataattgcatttcttcaggaaagtttgagggagaaaaaggtaaagaaaaatttagaaacaacttcagaataaagagattttctcttgggttacagagattgtcatatgacaaattataagcagacacttgagaaaactgaaggcccatgcctgcccaaattaccctttgaccccttggtcaagctgcaactttggttaaagggagtgtttatgtgttatagtgttcatttactcttctggtctaacccattggctccgtcttcatcctgcagtgacctcagtgcctcagaaacatacatatgtttgtctagtttaagtttgtgtgaaattctaactagcgtcaagaactgagggccctaaactatgctaggaatagtgctgtggtgctgtgataggtacacaagaaatgagaagaaactgcagattctctgcatctccctttgccgggtctgacaacaaagtttccccaaattttaccaatgcaagccatttctccatatgctaactactttaaaatcatttggggcttcacattgtctttctcatctgtaaaaagaatggaagaactcattcctacagaactccctatgtcttccctgatgggctagagttcctctttctcaaaaattagccattattgtatttccttctaagccaaagctcagaggtcttgtattgcccagtgacatgcacactggtcaaaagtaggctaagtagaagggtactttcacaggaacagagagcaaaagaggtgggtgaatgagagggtaagtgagaaaagacaaatgagaagttacaacatgatggcttgttgtctaaatatctcctagggaattattgtgagaggtctgaatagtgttgtaaaataagctgaatctgctgccaacattaacagtcaagaaatacctccgaataactgtacctccaattattctttaaggtagcatgcaactgtaatagttgcatgtatatatttatcataatactgtaacagaaaacacttactgaatatatactgtgtccctagttctttacacaataaactaatctcatcctcataattctattagctaatacatattatcatcctatatttcagagacttcaagaagttaagcaacttgctcaagatcatctaagaagtaggtggtatttctgggctcatttggcccctcctaatctctcatggcaacatggctgcctaaagtgttgattgccttaattcatcagggatgggctcatactcactgcagaccttaactggcatcctcttttcttatgtgatctgcctgaccctagtagacttatgaaatttctgatgagaaaggagagaggagaaaggcagagctgactgtgatgagtgatgaaggtgccttctcatctgggtaccagtggggcctctaagactaagtcactctgtctcactgtgtcttagccagttccttacagcttgccctgatgggagatagagaatgggtatcctccaacaaaaaaataaattttcatttctcaaggtccaacttatgttttcttaatttttaaaaaaatcttgaccattctccactctctaaaataatccacagtgagagaaacattcttttcccccatcccataaatacctctattaaatatggaaaatctgggcatggtgtctcacacctgtaatcccagcactttgggaggctgaggtgggtggactgcttggagctcaggagttcaagaccatcttggacaacatggtgataccctgcctctacaaaaagtacaaaaattagcctggcatggtggtgtgcacctgtaatcccagctattagggtggctgaggcaggagaattgcttgaacccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaatatatatatatatatacacatatacgtatatatatatatatatatatatttgttgttatcaattgccatagaatgattagttattgtgaatcaaatatttatcttgcaggtggcctctatacctagaagcggcagaatcaggctttattaatacatgtgtatagatttttaggatctatacacatgtattaatatgaaacaaggatatggaagaggaaggcatgaaaacaggaaaagaaaacaaaccttgtttgccattttaaggcacccctggacagctaggtggcaaaaggcctgtgctgttagaggacacatgctcacatacggggtcagatctgacttggggtgctactgggaagctctcatcttaaggatacatctcaggccagtcttggtgcattaggaagatgtaggcaactctgatcctgagaggaaagaaacattcctccaggagagctaaaagggttcacctgtgtgggtaactgtgaaggactacaagaggatgaaaaacaatgacagacagacataatgcttgtgggagaaaaaacaggaggtcaaggggatagagaaggcttccagaagaatggctttgaagctggcttctgtaggagttcacagtggcaaagatgtttcagaaatgtgacatgacttaaggaactatacaaaaaggaacaaatttaaggagaggcagataaattagttcaacagacatgcaaggaattttcagatgaatgttatgtctccactgagcttcttgaggttagcagctgtgagggttttgcaggcccaggacccattacaggacctcacgtatacttgacactgttttttgtattcatttgtgaatgaatgacctcttgtcagtctactcggtttcgctgtgaatgaatgatgtcttgtcagcctacttggtttcgctaagagcacagagagaagatttagtgatgctatgtaaaaacttcctttttggttcaagtgtatgtttgtgatagaaatgaagacaggctacatgatgcatatctaacataaacacaaacattaagaaaggaaatcaacctgaagagtatttatacagataacaaaatacagagagtgagttaaatgtgtaataactgtggcacaggctggaatatgagccatttaaatcacaaattaattagaaaaaaaacagtggggaaaaaattccatggatgggtctagaaagactagcattgttttaggttgagtggcagtgtttaaagggtgatatcagactaaacttgaaatatgtggctaaataactagaatactctttattttttcgtatcatgaatagcagatatagcttgatggccccatgcttggtttaacatccttgctgttcctgacatgaaatccttaatttttgacaaaggggctattcattttcattttatattgggcctagaaattatgtagatggtcctgaggaaaagtttatagcttgtctatttctctctctaacatagttgtcagcacaatgcctaggctataggaagtactcaaagcttgttaaattgaattctatccttcttattcaattctacacatggaggaaaaactcatcagggatggaggcacgcctctaaggaaggcaggtgtggctctgcagtgtgattgggtacttgcaggacgaagggtggggtgggagtggctaaccttccattcctagtgcagaggtcacagcctaaacatcaaattccttgaggtgcggtggctcactcctgtaatcacagcagtttgggacgccaaggtgggcagatcacttgaggtcaggagttggacaccagcccagccaacatagtgaaacctggtctctgcttaaaaatataaaaattagctggacgtggtgacgggagcctgtaatccaactacttgggaggctgaggcaggagaatcgcttgaaccggggaggtggagtttgcactgagcagagatcatgccattgcactccagcctccagagcgagactctgtctaaagaaaaacgaaaacaaacaaacaaacaaacaaacaaaacccatcaaattccctgaccgaacagaattctgtctgattgttctctgacttatctaccattttccctccttaaagaaactgtgaacttccttcagctagaggggcctggctcagaagcctctggtcagcatccaagaaatacttgatgtcactttggctaaaggtatgatgtgtagacaagctccagagatggtttctcatttccatatccacccacccagctttccaattttaaagccaattctgaggtagagactgtgatgaacaaacaccttgacaaaattcaacccaaagactcactttgcctagcttcaaaatccttactctgacatatactcacagccagaaattagcatgcactagagtgtgcatgagtgcaacacacacacacaccaattccatattctctgtcagaaaatcctgttggtttttcgtgaaaggatgttttcagaggctgaccccttgccttcacctccaatgctaccactctggtctaagtcactgtcaccaccacctaaattatagctgttgactcataacaatcttcctgcttctaccactgccccactacaatttcttcccaatatactatccaaattagtcttttcaaaatgtaagtcatatatggtcacctctttgttcaaagtcttctgatagtttcctatatcatttataataaaaccaaatccttacaattctctacaatagttgttcatgcatatattatgtttattacagatacatatatatagctctcatataaataaatatatatatttatgtgtatgtgtgtagagtgttttttcttacaactctatgatgtaggtattattagtgtcccaaattttataatttaggacttctatgatctcatcttttattctccccttcaccgaatctcatcctacattggccttattgatattccttgaaaattctaagcatcttacatctttagggtatttacatttgccattccctatgccctaaatatttaatcatagtttcatataaatgggttcctcatcatctatgggtactctctcaggtgttaactttatagtgaggactttcctgccatactacttaaagtagcgataccctttcaccctgtcctaatcacactctggccttcatttcagttttttttttttctccatagcacctaatctcattggtatataacatgtttcatttgcttatttaatgtcaagctctttccactatcaagtccatgaaaacaggaactttattcctctattctgtttttgtgctgtattcttagcaattttacaattttgaatgaatgaatgagcagtcaaacacatatacaactataattaaaaggatgtatgctgacacatccactgctatgcacacacaaagaaatcagtggagtagagctggaagtgctaagcctgcatagagctagttagccctccgcaggcagagccttgatgggattactgagttctagaattggactcatttgttttgtaggctgagatttgctcttgaaaacttgttctgaccaaaataaaaggctcaaaagatgaatatcgaaaccagggtgttttttacactggaatttataactagagcactcatgtttatgtaagcaattaattgtttcatcagtcaggtaaaagtaaagaaaaactgtgccaaggcaggtagcctaatgcaatatgccactaaagtaaacattatttcataggtgtcagatatggcttattcatccatcttcatgggaaggatggccttggcctggacatcagtgttatgtgaggttcaaaacacctctaggctataaggcaacagagctccttttttttttttctgtgctttcctggctgtccaaatctctaatgataagcatacttctattcaatgagaatattctgtaagattatagttaagaattgtgggagccattccgtctcttatagttaaatttgagcttcttttatgatcactgtttttttaatatgctttaagttctggggtacatgtgccatggtggtttgctgcacccatcaacccgtcatctacattaggtatttctcctaatgctatccttcccctagccccccacccccaacaggccccagtgtgtgatgttcccctccctgtgtccatggatcactggtttttttttgttttttttttttttttaaagtctcagttaaatttttggaatgtaatttattttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgagtacagaaaagaggctctagcagcatagtggtttagaggagatgtttctttccttcacagatgccttagcctcaataagcttgcggttgtggaagtttactttcagaacaaactcctgtggggctagaattattgatggctaaaagaagcccgggggagggaaaaatcattcagcatcctcacccttagtgacacaaaacagagggggcctggttttccatatttcctcatgatggatgatctcgttaatgaaggtggtctgacgagatcattgcttcttccatttaagccttgctcacttgccaatcctcagttttaaccttctccagagaaatacacattttttattcaggaaacatactatgttatagtttcaatactaaataatcaaagtactgaagatagcatgcataggcaagaaaaagtccttagctttatgttgctgttgtttcagaatttaaaaaagatcaccaagtcaaggacttctcagttctagcactagaggtggaatcttagcatataatcagaggtttttcaaaatttctagacataagattcaaagccctgcacttaaaatagtctcatttgaattaactctttatataaattgaaagcacattctgaactacttcagagtattgttttatttctatgttcttagttcataaatacattaggcaatgcaatttaattaaaaaaacccaagaatttcttagaattttaatcatgaaaataaatgaaggcatctttacttactcaaggtcccaaaaggtcaaagaaaccaggaaagtaaagctatatttcagcggaaaatgggatatttatgagttttctaagttgacagactcaagttttaaccttcagtgcccatcatgtaggaaagtgtggcataactggctgattctggctttctactcctttttcccattaaagatccctcctgcttaattaacattcacaagtaactctggttgtactttaggcacagtggctcccgaggtcagtcacacaataggatgtctgtgctccaagttgccagagagagagattactcttgagaatgagcctcagccctggctcaaactcacctgcaaacttcgtgagagatgaggcagaggtacactacgaaagcaacagttagaagctaaatgatgagaacacatggactcatagagggaaacaacgcatactggggcctatcagagggtggagggtgagagaaggagaggatcaggaaaaatcactaatggatgctaagcgtaatacctgagtgatgagatcatctatacaacaaacccccttgacattcatttatctatgtaacaaacctgcacatcctgtacatgtacccctgaacttaaaataaaagttgaaaacaagaaagcaacagtttgaacacttgttatggtctattctctcattctttacaattacactagaaaatagccacaggcttcctgcaaggcagccacagaatttatgacttgtgatatccaagtcattcctggataatgcaaaatctaacacaaaatctagtagaatcatttgcttacatctatttttgttctgagaatatagatttagatacataatggaagcagaataatttaaaatctggctaatttagaatcctaagcagctcttttcctatcagtggtttacaagccttgtttatatttttcctattttaaaaataaaaataaagtaagttatttgtggtaaagaatattcattaaagtatttatttcttagataataccatgaaaaacattcagtgaagtgaagggcctactttacttaacaagaatctaatttatataatttttcatactaatagcatctaagaacagtacaatatttgactcttcaggttaaacatatgtcataaattagccagaaagatttaagaaaatattggatgtttccttgtttaaattaggcatcttacagtttttagaatcctgcatagaacttaagaaattacaaatgctaaagcaaacccaaacaggcaggaattaatcttcatcgaatttgggtgtttctttctaaaagtcctttatacttaaatgtcttaagacatacatagattttattttactaattttaattatatagacaataaatgaatattcttactgattactttttctgactgtctaatctttctgatctatcctggatggccataacacttatctctctgaactttgggcttttaatataggaaagaaaagcaataatccatttttcatggtatctcatatgataaacaaataaaatgcttaaaaatgagcaggtgaagcaatttatcttgaaccaacaagcatcgaagcaataatgagactgcccgcagcctacctgacttctgagtcaggatttataagccttgttactgagacacaaacctgggcctttcaatgctataacctttcttgaagctcctccctaccacctttagccataaggaaacatggaatgggtcagatccctggatgcaagccaggtctggaaccataggcagtaaggagagaagaaaatgtgggctctgcaactggctccgagggagcaggagaggatcaaccccatactctgaatctaagagaagactggtgtccatactctgaatgggaagaatgatgggattacccatagggcttgttttagggagaaacctgttctccaaactcttggccttgagatacctggtccttattccttggactttggcaatgtctgaccctcacattcaagttctgaggaagggccactgccttcatactgtggatctgtagcaaattccccctgaaaacccagagctgtatcttaattggttaaaaaaaattatattatctcaacgactgttcttctctgagtagccaagctcagcttggttcaagctacaagcagctgagctgctttttgtctagtcattgttcttttatttcagtggatcaaatacgttctttccaaacctaggatcttgtcttcctaggctatatattttgtcccaggaagtcttaatctggggtccacagaacactagggggctggtgaagtttatagaaaaaaaatctgtatttttacttacatgtaactgaaatttagcattttcttctactttgaatgcaaaggacaaactagaatgacatcatcagtacctattgcatagttataaagagaaaccacagatattttcatactacaccataggtattgcagatctttttgtttttgtttttgtttgagatggagtttcgctcttattgcccaggctggagtgcagtggcatgatttcggctcactgcaacctccccttcctgcattcaagcaattctcctgccttggcctcctgagtagctggggattacaggcacctgccaccatgccagtctaatttttgtatttttagtagagatggggtttcgccatgttggccaggctggtcttgaactcctgacctcagatgatctgcccgccttggcctcctgaagtgctgggattataggtgtgagccaccacgcctggcccattgcagatatttttaattcacatttatctgcatcactacttggatcttaaggtagctgtagacccaatcctagatctaatgctttcataaagaagcaaatataataaatactataccacaaatgtaatgtttgatgtctgataatgatatttcagtgtaattaaacttagcactcctatgtatattatttgatgcaataaaaacatatttttttagcacttacagtctgccaaactggcctgtgacacaaaaaaagtttaggaattcctggttttgtctgtgttagccaatggttagaatatatgctcagaaagataccattggttaatagctaaaagaaaatggagtagaaattcagtggcctggaataataacaatttgggcagtcattaagtcaggtgaagacttctggaatcatgggagaaaagcaagggagacattcttacttgccacaagtgtttttttttttttttttttttatcacaaacataagaaaatataataaataacaaagtcaggttatagaagagagaaacgctcttagtaaacttggaatatggaatccccaaaggcacttgacttgggagacaggagccatactgctaagtgaaaaagacgaagaacctctagggcctgaacatacaggaaattgtaggaacagaaattcctagatctggtggggcaaggggagccataggagaaagaaatggtagaaatggatggagacggaggcagaggtgggcagatcatgaggtcaagagatcgagaccatcctggcaaacatggtgaaatcccgtctctactaaaaataaaaaaattagctgggcatggtggcatgcgcctgtagtcccagctgctcgggaggctgaggcaggagaatcgtttgaacccaggaggcgaaggttgcagtgagctgagatagtgccattgcactccagtctggcaacagagtgagactccgtctcaaaaaaaaaaaaaaaagaaagaaagaaaagaaaaagaaaaaagaaaaaataaatggatgtagaacaagccagaaggaggaactgggctggggcaatgagattatggtgatgtaagggacttttatagaattaacaatgctggaatttgtggaactctgcttctattattcccccaatcattacttctgtcacattgatagttaaataatttctgtgaatttattccttgattctaaaatatgaggataatgacaatggtattataagggcagattaagtgatatagcatgagcaatattcttcaggcacatggatcgaattgaatacactgtaaatcccaacttccagtttcagctctaccaagtaaagagctagcaagtcatcaaaatggggacatacagaaaaaaaaaaggacactagaggaataatataccctgactcctagcctgattaatatatcgattcacttttttctctgtttgatgacaaattctggctttaaataattttaggattttaggcttctcagctcccttcccagtgagaagtataagcaggacagacaggcaagcaagaagagagccccaggcaatactcacaaagtagccaatgtcccctgtggtcatagagaaatgaaaagagagaggattctctggaagcactggatgtaatcttttctgtctgtcctctctagggaatcaccccaaggtactgtactttgggattaaggctttagtcccactgtggactacttgctattctgttcagtttctagaaggaactatgtacggtttttgtctccctagagaaactaaggtacagaagttttgtttacaatgcactccttaagagagctagaactgggtgagattctgttttaacagctttattttcttttccttggccctgtttttgtcactgtcaccacctttaaggcaaatgttaaatgcgctttggctgaaactttttttcctattttgagatttgctcctttatatgaggctttcttggaaaaggagaatgggagagatggatatcattttggaagatgatgaagagggtaaaaaaggggacaaatggaaatttgtgttgcagatagatgaggagccaacaaaaaagagcctcaggatccagcacacattatcacaaacttagtgtccatccatcactgctgaccctctccggacctgactccacccctgagggacacaggtcagccttgaccaatgacttttaagtaccatggagaacagggggccagaacttcggcagtaaagaataaaaggccagacagagaggcagcagcacatatctgcttccgacacagctgcaatcactagcaagctctcaggcctggcatcatggtgcattttactgctgaggagaaggctgccgtcactagcctgtggagcaagatgaatgtggaagaggctggaggtgaagccttgggcaggtaagcattggttctcaatgcatgggaatgaagggtgaatattaccctagcaagttgattgggaaagtcctcaagattttttgcatctctaattttgtatctgatatggtgtcatttcatagactcctcgttgtttacccctggacccagagattttttgacagctttggaaacctgtcgtctccctctgccatcctgggcaaccccaaggtcaaggcccatggcaagaaggtgctgacttcctttggagatgctattaaaaacatggacaacctcaagcccgcctttgctaagctgagtgagctgcactgtgacaagctgcatgtggatcctgagaacttcaaggtgagttcaggtgctggtgatgtgattttttggctttatattttgacattaattgaagctcataatcttattggaaagaccaacaaagatctcagaaatcatgggtcgagcttgatgttagaacagcagacttctagtgagcataaccaaaacttacatgattcagaactagtgacagtaaaggactactaacagcctgaattggcttaacttttcaggaaatcttgccagaacttgatgtgtttatcccagagaattgtattatagaattgtagacttgtgaaagaagaatgaaatttggcttttggtagatgaaagtccatttcaaggaaatagaaatgccttattttatgtgggtcatgataattgaggtttagaaagagatttttgcaaaaaaaataaaagatttgctcaaagaaaaataagacacattttctaaaatatgttaaatttcccatcagtattgtgaccaagtgaaggcttgtttccgaatttgttggggattttaaactcccgctgagaactcttgcagcactcacattctacatttacaaaaattagacaattgcttaaagaaaaacagggagagagggaacccaataatactggtaaaatggggaagggggtgagggtgtaggtaggtagaatgttgaatgtagggctcatagaataaaattgaacctaagctcatctgaattttttgggtgggcacaaaccttggaacagtttgaggtcagggttgtctaggaatgtaggtataaagccgtttttgtttgtttgtttgttttttcatcaagttgttttcggaaacttctactcaacatgcctgtgtgttattttgtcttttgcctaacagctcctgggtaacgtgatggtgattattctggctactcactttggcaaggagttcacccctgaagtgcaggctgcctggcagaagctggtgtctgctgtcgccattgccctggcccataagtaccactgagttctcttccagtttgcaggtgttcctgtgaccctgacaccctccttctgcacatggggactgggcttggccttgagagaaagccttctgtttaataaagtacattttcttcagtaatcaaaaattgcaattttatcttctccatcttttactcttgtgttaaaaggaaaaagtgttcatgggctgagggatggagagaaacataggaagaaccaagagcttccttaagaaatgtatgggggcttgtaaaattaatgtggatgttatgggagaattccaggattccaaggaggatgatatgatggagaaaaatctttatcggggtgggaaaatggttaattaagtggacagagactcctaggcagtttttactgcaccggggaaagaaggagctgttagtggtacctgagaaagcagatttgtggtacatgtcacttttcattaaaaacaaaaacaaaacaaaacaaaacttcatagatatccaagatataggctagaattactattttaatttactcttatttacattttgaagtagctagcttgtcacatgttttatgaaattgatttggagataagatgagtgtgtatcaacaatagcctgctctttccatgaaggattccattatttcatgggttagctgaagctaagacacatgatatcattgtgcattatcttctgatagaatgtaacatgcactaaaataaagttagagttaggacctgagtgggaaagtttttggagagtgtgatgaagactttccgtgggagatagaatactaataaaggcttaaattctaaaaccagcaagctagggcttcgtgacttgcatgaaactggctctctggaagtagaagggagagtaagacatacgtagaggactaggaaagaccagatagtacagggcctggctacaaaaatacaagcttttactatgctattgcaatactaaacgataagcattaggatgttaagtgactcaggaaataagattttgggaaaaagtaatctgcttatgtgcacaaaatggattcaagtttgcagataaaataaaatatggatgatgattcaaggggacagatacaatggttcaaacccaagaggagcagtgagtctgtggaatttgaaggatggacaaaggtggggtgagaaagacatagtattcgactgactgtgggagatgagaaggaagaaggaggtgataaatgactgaaagctcccagactggtgaagataacaggaggaaaccatgcactgacctggtgactctcatgtgtgaagggtagagggatattaacagatttactttttaggaagtgctagattggtcagggagttttgaccttcaggtcttgtgtctttcatatcaaggaacctttgcattttccaagttagagtgccatattttggcaaatataactttattagtaattttatagtgctctcacattgatcagactttttcctgtgaattacttttgaatttggctgtatatatccagaatatgggagagagacaaataattattgtagttgcaggctatcaacaatactggtctctctgagccttataacctttcaatatgcccataaacagagtaaacagggattattcatggcactaaatattttcacctagtcagtcaacaaatgggagcaatgtgcattttttgatacatatttttatatatttatggggtacatgtgatacttacatgcctagaacatgtgatgattaagtctagatatttaggatatccattgctttgagcatttatcatttctatgtattgagaaaatttcaaatcctcatttctagccattttgaaatatataataaatagtaattaactatagtcaccctactcaaatatcaaacattatggcttaatccttctatccaactgtgtttgtacctattaaccaacatctcttaaatcccctcccatacacactcacactttttccagcctctgataactatcattctactctctaccaccatgagacccacttttttagctcccacagatgaataaaaacatgtgatatttgactttctgtatctggcttattttattatctatctctttggcataccaagagtttgtttttgttctgcttcagggctttcaattaacataatgacctctggttccatccatgttgctacaaatgacaagatttcattctttttcatggcaaaatagtactgtgcaaaaatacaattttttaatccgttcatctgttgatagacacttaggttgatcccaaaccttaactattgtgaatagtgcttcaataaacatgagtgtaatgtgtccattggatatactgatttcctttcttttggataaataaccactagtgagattgctggattgtatgatagttctgtttttagtttactgagaaatcttcatactgttttccataatggttgtactattttacattcccaccaacagtgtgtaagaaagagttcccttttctccatatcctcacaaggatctgttattttttgtcttttttgttaatagccgttttaactagagtaagtagatatctcattgtagttttgatttgcatttccctgatcattagtgatgttgagaattttttcatatgtttgttggtcatttgtatatctttttctgagaattgtctgttcatgtccttagcctactttttattgggattgtttgttattttcttgataatctatttgtgttcattttagagcctggatattattcttttgtcagatgtatagattgtgaagattttctcccactctgtgggttgtctgtttattctgcagactcttccttttgccatgcaaaagctctttagtttaatttagtcccagatattttctttgtttttatgtatttgcatttgtgttcttggtcatgaaatcctttcctaagccaatgtgtagaagggtttttccgatgttattttctagaattgttacagtttcagggcttagatttaagtccttgatccatcttgagttgatttttgtataaggtgagagatgaagatccagtttcattctcctacatgtagcttgccagctatccccgcaccatttgttgaatagggtgccctttccccactttatgtttttgtttgctttgtcaaagatcagttggatgtaagtatttgagtttatttctgggttctctattctgttccattggtcgatgtgcctatttgtacaccagcatcatgctgttttggtgactatggccttattgtatagtttgaaatgaggtaatgtaatgccttcagatttgttcttttttttagacttgcttgtttattgggctcttttttggttccataagaattttaggattgttttttctagttctgtgaagactaatggtggtattttgatgggaattgcaatgaatttgtaggttgcttctggcattatggccattttcacaatattgattctacccatctatgagaatggcatgtgtttccatttgtttgtgtcttatatgattactttcagccgtgttttgtagttttccttgtagatgtctttcacctccttggttaggtatatattcctaagtttttgttttgttttgttttgttttttgcagctattgtaaaaggggttgagttcttgattttattctcagcttggtcattgctggtatgtaagaaagcaactcattggtgtacgttaattttgtatccagaaactttgctgaattattttatcagttctagggggttttggaggagtctttagagttttctacatacacaatcatatcatcagcaaacagtgacagtttgactttctctttaacaatttggatgtgctttacttgtttctcttgtctgattgctcttgctaggacttccagtaatatgttaaagagaagtggtgagagtgggtatccttgtctcattccagttttcagacagaatgcttttaactttttcccattcaatataatgttggctgtgtgtttaccatagctggcttttattacattgaggtatgtcctttgtaaaccgattttgctgagttttagtcataaagtgatgttgaattttgttgaatgcagtttctgtggctattgagataatcacatgatttttgtttccaattctctttatgttgtgtatcacacttattgacttgcgtatgttaaaccatccgtgcatccctcgcatgaaacccacttgatcatgggttttgatatgctgtcggatgctattagctagtattttgtcaaggatgttggcatctatgttcatcagggatattgatctgtagtgttttttttttttggttatgttctttcccagttttggtattaaggtgatactggcttcatagaatgatttagggaggattctctctttctctatcttgtagaatactgtcaataggattggtatcaattcttctttgaatgtctggtagaattcagctgtgaatctatctggtcctggacttttttgttgttggtaaatttttattatcatttcagtcttgctgcttattactggtctgttcagggtatctaattcttcctgacttaagctagagccctgtatctttccaggaattcgaacgtctcctttaggttttctagtttatgcatgtaaaggtgttcatagtagccttgaataatcttttgtatttctgtggtatcagtaatagtatctcctgttttgtttctaattgagtttatttgcacttctctcctcttttcttggttaatcttgctaatggtctatcagttttatttatcttttcaaagaaccagctttttatttcatttagcttttgtatttttttgcagttgttttaatttcatttagttctcctcttatcttagttattccctttcttttgctgggttttggttctgtttgtttttgtttctctagtttcttgtggtgtgaccttatattgtctgtctgtcctctttcagactctttgacatcgacatttagggctgtgaactttccttttagcaccatctttgctgtatcctagaggttttgataggttgtgtcactattgtcggtcagttcaagtaattttgttgttcttattatactttaagttctgggatacatgtgcagaatgtgcaggtttgttacataggtatagatgtgccatggtggtttgctgcacccatcaacctgtcatctacattaggtatttcttttaatgttatccctctcctaaccccctcaccccccgacaggccctggtgtgtgatgttcccctccctgtgtccatgtgttctcattgttcaactcccacttatgagtgagaacgtgtggtgtttggtttctctgttcctgtgttagtttgctcagaatgatggtttccaccttcatccatgtccctgcaaagacatgaactcatcatttttatggctgcatagtattccatggtgtatatgtgccacattttctttatccattatatcgctgatggccatttgggttggttccaagtctttgctattgtgaatagtgccacaataaacatacgtgtgcacgtgtctttatagtagaatgatttctaattctttgggtatatacccagtaatgggattgctgggtcaaacagtatttctggttctagatccttgaggaatcgccacactgtcttccacaatggttgaactaatttacacacccatcaacagtgtaaaatttttcctattcttccacatcctctccagcaccttttgtttcctgactttttaataattgccattctaactggcatgagatggtatctcattgtggttttgatttgcatttctctaatgaccagtgatgatgagcttcttttcatgtgtttcttggccacataaatgacttctttagagaagcatctgttcatatcctttgtccactttttgatggggtcgttaggttttttcttgtaaatttgttgaagttctttgtagattttggatgttagccctttgtcagatggatagattgcaaaaattttctcccattctgtaggttgcctgttcactctgatgatagtcttttgctgtgcagaagctctttagtttaattagatcccatatgtcaattttggcctttgttgtcattgcttttgatgttttagtcgtgaatttttgcccatgcctatgtcctgaatggtattgcctaggttatcttctaggatttttatggttttaggttgcacatttaagtctttaatccaccttgagttaatttttgtataaggtgtaaggaaggggtacagtttcagttttatgcatattgctagccagtttttccagcaccatttattaaatagggaattctttctccattgcttttgtgatgtttgtcaaagatcagatggtcgtagatgtgtggcattatttctgaggcttctgttctgttccactggtctatatatctgttttggtaccagtaccatgctgtttttgttactgtagccttgtagtatagtttgaagtcaggtagcatcatgcctccagctttgttctttttgtttaggattgtcttggctatatgggctcttttttgattccatatgacatttaaagtagttttttctaattctttgaaaaaagtcagtggtagcttgatggggatagcattgaatctataaattactttgggcagtatggccattttaaagatattgattctttctatctatgagcatggaatgtttttccatttgtttgtgtcctctcttatttccttgagcagtgagtggtttgtagctctccttgaagaggttcttcacatcccttagaagttgtatttctaggtattttattttattctctttgcagcaattgtgaatgggagttcacccatgatttggctctctgcttgtctattattggtgtataggaacgcttgtgatttctgcacactgattttgtatcttgagactttgctgaagctgtttatcagcttaagattttgggctgagatgacagggtcttctaaatatacaatcatgtcatctgcaaacagagacaatttgacttcctctcttcctatttgaatatgctttatttctttctcttgcctgattgtcctggcgagaacttccaatactatgttgagtaagagtggcgagagggcatccttgtcttgtgccggttttcaaagcaaatgatttttaaatttccatcttgatttcattgttgacccaatgatcattcaggagcaggttatttaatttccctgtatttgcatggttttgaaggttccttttgtagttgatttccaattttattctactgtggtctgagagagtgcttgatataatttcaatttttaaaaatttattgaggcttgttttgtggcatatcatatggcctatcttggagaaagttccatgtgctgatgaatagaatgtgtattctgcagttgttgggtagaatgtcctgtaaatatctgttaagtccatttgttctttaaatccattgtttctttgtagactgtcttgatgacctgcctagtgcagtcagtggagtattgaagtcccccactattattatgttgctgtctagtctagtagtaattgttttataaatttgggatctccagtattagatgcatatatattaagaattgtaatattctcccattggacaagggcttttatcattatatgatgtccctctttgtcttttttaactgctgtttctttaaagtttgttttgtctgacataagaatagctgctttggctcgcttttggtgtccatttgtgtggaatgtcattttccacccctttaccttaagtttatgtgagtccttatgtgttaggtgagtctcctgaaggcggcagataactggttggtgaattcttattcattctgcaattctgtatcttttaagtggagcatttagtccatttacattcaacatcagtattgaggtgtgaggtactattccattcttcgtggtatttgttgcctgtgtatctttttatctgtatttttgttgtatatgtcctatgggatttatgctttaaagaggttctgttttgatgtgcttccagggtttatttcaagatttagagctccttttatcagttcttgtagtgttggcttggtagtgccgaattctctcagcatttgtttttctgaaaaacactgtgtattttcttcatttgtgaagcttagtttcactggatataaaattcttggctgataattgttttgtttaagaaggctgaagatagggccatattcacttctagcttttacggtttctgctgagaaatctgctgttaatctgataggttttctttcataggttacctggtagtttcacctcacagctcttaagattctctttgtctttagataactttggatactctgatgacaatgtacctaggcaatgatatttttgcaatgaatttcccaggtgtttattgagcttcttgtatttggatatctaggtctctagcaaggtgggggaagttttccttgattatttccctggataagttttccaaacttttagatttctcttctttctcaggaatgctgattattcttaggtttgattgtttaacataatcccagatttcttggaggctttgttcatattttcttattcttttttctttgtctttgttggattgggttaattcaaaaactttgtcttcaagctctgaatttcttctgcttggattctattgctgagactttctagagcattttgcatttctataagtgcatccattcatccattgtttcctgaagttttgaatgttttttatttatgctatctctttaactgaagatttctcccctcatttcttgtatcatatttttggtttttttaaaattggacttcaccttcctcggatgcctccttgattagcttaataactgaccttctgaattatttttcaggtaaatcagggatttcttcttggtttggatgcattgctggtgagctagtatgattttttggggggtgttaaagaaccttgtttttcatattaccagagttagttttctggttccttctcacttgggtaggctctgtcagagggaaagtctaggcctcaaggctgagacttttgtcccatgaggtgttcccttgatgtagcacagtcccccttttcctaggcgtggggcttcctgagagccgaactgtagtgattgttatctctcttctggatctagccacccatcaggtctaccagactccaggctggtactggggtttgtctgcacagagtcttgtgacgtgaaccatctgtgggtctctcagccatagatacaaccacctgctccaatggaggtggcagaggatgaaatggactctgtgagggtccttacttttggttgttcaatgcactatttttgtgctggttggcctcctgccaggaggtggcactttctagaaagcatcagcagaggcagtcaggtggtggtggctgggggggctggggcaccctagaactcccaagaatatatgccctttgtcttcagctaccagggtgagtaaggaaggaccatcaggtgggggcaggactagtcgtgtctgagctcagagtctccttgggcaggtctttctgtggctactgtgggaggatgggggtgtagtttccaggtcaatggatttatgttcctaggacaattatggctgcctctgctgtgtcatgcaggtcatcaggaaagtgggggaaagcaagcagtcacgtgacttgcccagctcccatgcaactcaaaaggttggtctcacttccagcgtgcaccctcccccgcaacagcaccgaatctgtttccatgcagtcagtgagcaaggctgagaacttgccccaggctaccagctgcgaaaccaagtagggctgtcctacttccctgccagtggagtctgcacaccaaattcatgtccccccaccaacccccccactgcccagcccctagatctggccaggtggagattttctttttcctgtcatcttttcccagttcctctggcagccctcccaaatgacccctgtgaggcaaggcagaaatggcttcctaggggacccagagagcccacagggcttttcccgctgcttcctctacccctgtattttgcttggccctctaaattgactcagctccaggtaaggtcagaatcttctcctgtggtctagatcttcaggttccccagtgaggatgtgtgtttgggggtagacggtcccccttttccacttccacagtttgggcactcacaatatttggggtgtttcccgggtcctgcaggagcaatctgcttctttcagagggtgtgtgcgttctctcagctttcttgatttatttctgcaggtggttctgcaaaaaaaattcctgatgggagacttcacatgctgctctgtgcatccgagtgggagctgcaatgtacttctgctgcctcccatctgccatcaccctctaatttgtcggtaatatgcatttttaatcaatctttttttctctctctctctttttcttctcccccaaaactatactgccctttgatatcaaggaatcaaggacgtgatgttgaggggtgggcagtggatacactctttaccccttagggagctatatctagatttagatattgccaattcaagataacttaattgaaagcaaattcataatgaatacacacacacacacacacatctgcatgacaagatttttaatagttgaaagaataactaataattgtccacaggcaataagggctttttaagcaaaacagttgtgataaacaggtcattcttagaatagtaatccagccaatagtacaggttgcttagagattatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaagggggctgaacccccttccctggattgcagcacagcagcgaggaaggggctcaacgaagaaaaagtgttccaagctttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccggtgggaagaaagacctctataggacaggacttctcagaaacagatgttttggaagagatgggaaaaggttcagtgaagacctgggggctggattgattgcagctgagtagcaaggatggttcttaaggaagggaaagtgttccaagctttaggaattcaaggtttagtcaggtgtagcaattctattttattaggaggaatactatttctaatggcacttagcttttcacagcccttgtggatgcctaagaaagtgaaattaatcccatgccctcaagtgtgcagattggtcacagcatttcaagggagagacctcattgtaagactctgggggaggtggggacttaggtgtaagaaatgaatcagcagaggctcacaagtcagcatgagcatgttatgtctgagaaacagaccagcactgtgagatcaaaatgtagtgggaagaatttgtacaacattaattggaaggcttacttaatggaatttttgtatagttggatgttagtgcatctctataagtaagagtttaatatgatggtgttacggacctaatgtttgtgtctcctcaaaattcacatgctgaatccccaactcccaactgaccttatctgtgggggaggcttttgaaaagtaattaggtttagatgagctcataagagcagatccccatcataaaattattttccttatcagaagcagagagacaagccatttctctttcctcccggtgaggacacagtgagaagtccgccatctgcaatccaggaagagaaccctgaccacgagtcagccttcagaaatgtgagaaaaaactctgttgttgaagccacccagtcttttgtattttgttatagcaccttgcactgagtaaggcagatgaagaaggagaaaaaaataagcttgggttttgagtggactacagaccatgtttatctcaggtttgcaaagctcccctcgtcccctatgtttcagtataaaatacctactctactactctcatctataagacccaaataataagcctgcgcccttctctctaactttgatttctcctatttttacttcaacatgctttactctagccttgtaatgtctttacatacagtgaaatgtaaagttctttattctttttttctttctttcttttttctcctcagcctcagaatttggcacatgcccttccttctttcaggaacttctccaacatctctgcctggctccatcatatcataaaggtcccacttcaaatgcagtcactaccgtttcagaatatgcactttctttcttttttgttttttgttttttttaagtcaaagcaaatttcttgagagagtaaagaaataaacgaatgactactgcataggcagagcagccccgagggccgctggttgttccttttatggttatttcttgatgatatgttaaacaagttttggattatttatgccttctctttttaggccatatagggtaactttctgacattgccatggcatttttcttttaatttaatttactgttaccttaaattcaggggtacacgtacaggatatgcaggtttgttttataggtaaaagtgtgccatggttttaatgggttttttttttcttgtaaagttgtttaagtttcttgtttactctggatattaggcctttgtcagaagaatagattggaaaatctttttcccattctgtagattgtctttcgctctgatggtagtttcttttgctgagcaggagctctttagtttaattagattccattggtcaatttttgcttttgctgcaattgcttttcacgctttcatcatgaaatctgtgcccgtgtttatatcatgaatagtattgccttgatttttttctaggctttttatagtttggggtttttcatttaagtctctaatccatctggagttaattttggataaggtataaggaaggagtccagtttcatttttcagcatatggctagccagttctcccccatcatttattaaattgaaaatcctttccccattgcttgcttttgtcaggtttctaaaagaccagatggttgtaggtacaatatgcagtttcttcaagtcatataataccatctgaaatctcttattaattcatttcttttagtatgtatgctggtctcctctgctcactatagtgagggcaccattagccagagaatctgtctgtctagttcatgtaagattctcagaattaagaaaaatggatggcatatgaatgaaacttcatggatgacatatggaatctaatatgtatttgttgaattaatgcataagatgcaacagagagaagttgacaactgcaatgataacctggtattgatgatataagagtctatagatcacagtagaagcaataatcatggaaaacaattggaaatggggaacagccacaaacaagaaagaatcaatacttccaggaaagtgactgcaggtcacttttcctggagcgggtgagagaaaagtggaagttagcagtaactgctgaattcctggttggctgatggaaagatggggcagctgttcactggtacgcagggttttagatgtatgtacctaaggatatgaggtatggcaatgaacagaaattcttttgggaatgagttttagggccattaaaggacatgacctgaagtttcctctgaggccagtccccacaactcaatataaatgtgtttcctgcatatagtcaaagttgccacttctttttcttcatatcatcgatctctgctcttaaagataatcttggttttgcctcaaactgtttgtcactacaaactttccccatgttcctaagtaaaacaggtaactgcctctcaactatatcaagtagactaaaatattgtgtctctaatatcagaaattcagctttaatatattgggtttaactctttgaaatttagagtctccttgaaatacacatgggggtgatttcctaaactttatttcttgtaaggatttatctcaggggtaacacacaaaccagcatcctgaacctctaagtatgaggacagtaagccttaagaatataaaataaactgttcttctctctgccggtggaagtgtgccctgtctattcctgaaattgcttgtttgagacgcatgagacgtgcagcacatgagacacgtgcagcagcctgtggaatattgtcagtgaagaatgtctttgcctgattagatataaagacaagttaaacacagcattagactatagatcaagcctgtgccagacacaaatgacctaatgcccagcacgggccacggaatctcctatcctcttgcttgaacagagcagcacacttctcccccaacactattagatgttctggcataattttgtagatatgtaggatttgacatggactattgttcaatgattcagaggaaatctcctttgttcagataagtacactgactactaaatggattaaaaaacacagtaataaaacccagttttccccttacttccctagtttgtttcttattctgctttcttccaagttgatgctggatagaggtgtttatttctattctaaaaagtgatgaaattggccgggcgcggtggctcacacctgtaatcccagcactttgggaggctgaggtgggcggatcacgaggtcaggagatcaagaccatcctggctaacatggtgaaaccccatctctactaaaaatacaaaaaattagccagagacagtggcgggtgcctgtagtcccagctactcgggaggctgaggcaggagaatggcgtgaacctgggaggcagagcttgcggtgagcagagatcgcgccactgcacactccagcctgggtgacaaagcgagactccatctcaaaaaaaaaaaaaaaaaaaagaaaaagaaagaaagaaagaaaaaaaaactgatgaaattgtgtattcaatgtagtctcaagagaattgaaaaccaagaaaggctgtggcttcttccacataaagcctggatgaataacaggataacacgttgttacattgtcacaactcctgatccaggaattgatggctaagatattcgtaattcttatccttttcagttgtaacttattcctatttgtcagcattcaggttattagcggctgctggcgaagtccttgagaaataaactgcacactggatggtgggggtagtgtaggaaaatggaggggaaggaagtaaagtttcaaattaagcctgaacagcaaagttcccctgagaaggccacctggattctatcagaaactcgaatgtccatcttgcaaaacttccttgcccaaaccccacccctggagtcacaacccacccttgaccaatagattcattttactgagggaggcaaagggctggtcaatagattcatttcactgggagaggcaaagggctgggggccagagaggagaagtaaaaagccacacatgaagcagcaatgcaggcatgcttctggctcatctgtgatcaccaggaaactcccagatctgacactgtagtgcatttcactgctgacaagaaggctgctgccaccagcctgtgaagcaaggttaaggtgagaaggctggaggtgagattctgggcaggtaggtactggaagccgggacaaggtgcagaaaggcagaaagtgtttctgaaagagggattagcccgttgtcttacatagtctgactttgcacctgctctgtgattatgactatcccacagtctcctggttgtctacccatggacctagaggtactttgaaagttttggatatctgggctctgactgtgcaataatgggcaaccccaaagtcaaggcacatggcaagaaggtgctgatctccttcggaaaagctgttatgctcacggatgacctcaaaggcacctttgctacactgagtgacctgcactgtaacaagctgcacgtggaccctgagaacttcctggtgagtagtaagtacactcacgctttcttctttacccttagatatttgcactatgggtacttttgaaagcagaggtggctttctcttgtgttatgagtcagctatgggatatgatatttcagcagtgggattttgagagttatgttgctgtaaataacataactaaaatttggtagagcaaggactatgaataatggaaggccacttaccatttgatagctctgaaaaacacatcttataaaaaattctggccaaaatcaaactgagtgtttttggatgagggaacagaagttgagatagagaaaataacatctttcctttggtcagcgaaattttctataaaaattaatagtcacttttctgcatagtcctggaggttagaaaaagatcaactgaacaaagtagtgggaagctgttaaaaagaggattgtttccctccgaatgatgatggtatacttttgtacgcatggtacaggattctttgttatgagtgtttgggaaaattgtatgtatgtatgtatgtatgtatgtgatgactggggacttatcctatccattactgttccttgaagtactattatcctactttttaaaaggacgaagtctctaaaaaaaaaatgaaacaatcacaatatgttggggtagtgagttggcatagcaagtaagagaaggataggacacaatgggaggtgcagggctgccagtcatattgaagctgatatctagcccataatggtgagagttgctcaaactctggtgaaaaaggatgtaagtgttatatctatttactgcaagtccagcttgaggccttctattcactatgtaccattttcttttttatcttcactccctccccagctcttaggcaacgtgatattgattgttttggcaacccacttcagcgaggattttaccctacagatacaggcttcttggcagtaactaacaaatgctgtggttaatgctgtagcccacaagaccactgagttccctgtccactatgtttgtacctatggtccactatgtttgtacctatgtcccaaaatctcatctcctttagatgggggaggttggggagaagagcagtatcctgcctgctgattcagttcctgcatgataaaaatagaataaagaaatatgctctctaagaaatatcattgtactctttttctgtctttatattttaccctgattcagccaaaaggacgcactatttctgatggaaatgagaatgttggagaatgggagtttaaggacagagaagatactttcttgcaatcctgcaagaaaagagagaactcgtgggtggatttagtggggtagttactcctaggaaggggaaatcgtctctagaataagacaatgtttttacagaaagggaggtcaatggaggtactctttggaggtgtaagaggattgttggtagtgtgtagaggtatgttaggactcaaattagaagttctgtataggctattatttgtatgaaactcaggatatagctcatttggtgactgcagttcacttctacttattttaaacaacatattttttattatttataatgaagtggggatggggcttcctagagaccaatcaagggccaaaccttgaactttctcttaacgtcttcaatggtattaatagagaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtacaagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctgagatttaggaaacagcacgttaagtcaagttgatagaggagaatatggacatttaaaagaggcaggatgatataaaattagggaaactggatgcagagaccagatgaagtaagaaaaatagctatcgttttgagcaaaaatcactgaagtttcttgcatatgagagtgacataataaatagggaaacgtagaaaattgattcacatgtatatatatatatagaactgattagacaaagtctaacttgggtatagtcagaggagcttgctgtaattatattgaggtgatggataaagaactgaagttgatggaaacaatgaagttaagaaaaaaaatcgagtaagagaccattgtggcagtgattgcacagaactggaaaacattgtgaaacagagagtcagagatgacagctaaaatccctgtctgtgaatgaaaagaaggaaatttattgacagaacagcaaatgcctacaagccccctgtttggatctggcaatgaacgtagccattctgtggcaatcacttcaaactcctgtacccaagacccttaggaagtatgtagcaccctcaaacctaaaacctcaaagaaagaggttttagaagatataataccctttcttctccagtttcattaatcccaaaacctctttctcaaagtatttcctctatgtgtccaccccaaagagctcacctcaccatatctcttgagtgggagcacatagataggcggtgctaccatctaacagcttctgaaattcctttgtcatatttttgagtccccactaataacccacaaagcagaataaataccagttgctcatgtacaataatcactcaactgctgtcttgtagcatacattaattaagcacattctttgaataattactgtgtccaaacaatcacactttaaaatctcacacttgtgctatcccttgcccttctgaatgtcactctgtattttaaatgaagagatgagggttgaatttcctgtgttacttattgttcatttctcgatgaggagttttcacattcacctttagtggaaaacacataagtacacatcttacaggaaaaatataccaaactgacatgtagcatgaatgcttgtgcatgtagtcatataaaatcttgtagcaatgtaaacattctctgatatacacatacagatgtgtctatatgtctacacaatttcttatgctccatgaacaaacattccatgcacacataagaacacacactgttacagatgcatacttgagtgcattgacaaaattaccccagtcaatctagagaatttggatttctgcatttgactctgttagctttgtacatgctgttcatttactctgggtgatgtctttccctcattttgccttgtctatcttgtactcatactttaagtcctaacttatatgttatctcaactaagaagctatttttttttaattttaactgggcttaaagccctgtctataaactctgctacaattatgggctctttcttataatatttagtgtttttcctactaatgtacttaatctgctcattgtatattcctaccactaaattttaacctcttttatggtagagacattgtcttgtaaactcttatttccctagtatttggagatgaaaaaaaagattaaattatccaaaattagatctctcttttctacattatgagtattacactatccatagagaagtttgtttgagacctaaactgaggaacctttggttctaaaatgactatgtgatatcttagtatttataggtcatgaggttccttcctctgcctctgctatagtttgattagtcaacaagcatgtgtcatgcatttattcacatcagaatttcatacactaataagacatagtatcagaagtcagtttattagttatatcagttagggtccatcaaggaaaggacaaaccattatcagttactcaacctagaattaaatacagctcttaatagttaattatccttgtattggaagagctaaaatatcaaataaaggacagtgcagaaatctagatgttagtaacatcagaaaacctcttccgccattaggcctagaagggcagaaggagaaaatgtttataccaccagagtccagaaccagagcccataaccagaggtccactggattcagtgagctagtgggtgctccttggagagagccagaactgtctaatgggggcatcaaagtatcagccataaaaaaccataaaaaagactgtctgctgtaggagatccgttcagagagagagagagaccagaaataatcttgcttatgctttccctcagccagtgtttaccattgcagaatgtacatgcgactgaaagggtgaggaaacctgggaaatgtcagttcctcaaatacagagaacactgagggaaggatgagaaataaatgtgaaagcagacatgaatggtaattgacagaaggaaactaggatgtgtccagtaaatgaataattacagtgtgcagtgattattgcaatgattaatgtattgataagataatatgaaaacacagaattcaaacagcagtgaactgagattagaattgtggagagcactggcatttaagaatgtcacacttagaatgtgtctctaggcattgttctgtgcatatatcatctcaatattcattatctgaaaattatgaattaggtacaaagctcaaataatttattttttcaggttagcaagaactttttttttttttttctgagatagagcattgctatggttgcccaggctggagtgcaatggcatgatccaggctcactgcaacatctgcctcccaggttcaagcgattctcctgcctcagcctcccaagtagctggcactacaggcatgtgccaccaccatgcctggctaattttctatttttagtagatagggggtttcaccatgttggtcaggctgatctcgaactcctaacatcaggtgatccaccctcctcggcctctgaaagtgctgggatcacaggcgtgagccaccacacccagccaagaatgtgaattttgtagaaggatataacccatatttctctgaccctagagtccttagtatacctcccataccatgtggctcatcctccttacatacatttcccatctttcaccctaccttttcctttttgtttcagcttttcactgtgtcaaaatctagaaccttatctcctacctgctctgaaaccaacagcaagttgacttccattctaacccacattggcattacactaattaaaatcgatactgagttctaaaatcatcggggattttggggactatgtcttacttcatacttccttgagatttcacattaaatgttggtgttcattaaaggtccttcatttaactttgtattcatcacactcttggattcacagttatatctaaactcttaaatacagcctgtataatcccaattcccaactctgatttctaacctctgacctccaacctcagtgccaaacccatatatcaaacaatgtactgggcttatttatatagatgtcctataggcacctcagactcagcatgggtatttcacttgttatactaaaactgtttctcttccagtgttttccattttagtcattagatagctacttgcccattcaccaaggtcacagattaaaatcatttccctacctctaatcaacagttcgattctgcttcaatttgtccctatctattaatcaccactcttactgcccagtcaggtcctcattgtttcctgaacaagagtagatgctattctttccacttttagaccttatcctggctggatgcggtggctcaggcttgtaaacccagcactttgggaggccaaggcaggcagatcacttgaggtcaggagttcaagaccagcctgaccaacatggtgaaaccccatctctactaaaaatacaaaatcagccgggcgtgtggtgcatgcctgcagtcccagctattcaggtggctgaggcaggagaattgcttgaacccaggaggcagaggttgcggtgagcctagattgcaccattgcactctagcttgggcaatagggatgaaactccatctcagaagagaaaagaaaaaaagaccttattctgttatacaaatcctctcaatgcaatccatatagaataaacatgtaaccagatctcccaatgtgtaaaatcatttcaggtagaacagaattaaagtgaaaagccaagtctttggaattaacagacaaagatcaaataacagtcctcatggccttaagaatttacctaacattttttttagaatcaattttcttatatatgaattggaaacataattcctccctcacaaacacattctaagattttaaggagatattgatgaagtacatcatctgtcatttttaacaggtagtggtagtgattcacacagcacattatgatctgttcttgtatgttctgttccattctgtattcttgacctggttgtattctttctgagctccagatccacatatctaagtacatctttttgcattttacaagagtgcatacaatacaatgtatccaagactgtatttctgattttatcgtaccactaaactcacaaatgtggccctattcttgtgttcacgactgacatcaccgtcatggtccaagtctgataatagaaatggcattgtcactttcttccctactgcaacagaagcccagctatttgtctcccattttctctacttctaaaatacatttcttcactaagtgagaataatcttttaaagacacaaatcaaaccatgccaccacctttcttgaattattcaatatctttcgttggcttccaggttacagaaaaataacttgtaacaaagtttaaaggtcattcatggctcctctctaccctattttataacatttccccttgtgatcagaatctcaggcacatcatccatctttctatatacaaataaagtcatatagtttgaactcacctctggttacttttaatcaaccaaatgctgtaaaatgcatttgtatcgctacgtgttaagcagtagttgattcttttcatttctgtgtaatattctattctttgactataccgtaatttatcaattctactgttggtaagcatttaagtggctaccggtttgaggtttttatgattattgctgtcataagcatttctatacatgtctttggatacacacatgcatgtgtttctgaatatctaaaaatgtaattgctaggtaatagacttatcaagcatccagcatttgtggatactattaaaggttttccaaaggggttatactattgtacagtgtcaccaacagagtttgagtttctattgatccatatcaccaccaaaatttgaactgtcagtcttatctcttctcttgtctcttttttcctcttttttttccttcccttcccctctcttcgtttcttttctctcctcttctcttctttcctctcttcccttccctttctctttctcttccctatcccttctcctctcctctcccctccttttttctcctctcctctccattatttatttttccttcttctcctccatcccttccatcctctctcttcccctcttccttccttcctttctccatttcttcctcctctttccttcaatccttccttttggatatgctcatgggtgtgtatttgtctgccattgtggcattatttgaattcagaaaagagtgaaaaactactgggatcttcattcctgggtctaattccacatttttttttaagaacacatctgtaaaaatgttctgtactagcatattcccaggaacttcgttaaatttaatctggctgaatatggtaaatctacttttcactttgcattctttctttagtcataccataattttaaacattcaaaatatttgtatataatatttgattttatctgtcattaaaatgttaaccttaaaattcatgtttccagaacctatttcaataactggtaaataaacactattcattttttaaatattcttttaatggatatttatttcaatataataaaaaattagagttttattataggaagaatttaccaaaagaaggaggaagcaagcaagtttaaactgcagcaatagatttgtccattccaacctctcaaaattcccttggagacaaaaatctctagaggcaaagaagaactttatattgagtcaacttgttaaaacatctgcttttagataagttttcttagtataaagtgacagaaacaaataagttaaactctaagatacattccactatattagcctaaaacacttctgcaaaaatgaaactaggaggatatttttagaaacaactgctgaaagagatgcggtggggagatatgtagaggagaacagggtttctgagtcaagacacacatgacagaacagccaatctcagggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcaggttggtatcaaggttataagagaggctcaaggaggcaaatggaaactgggcatgtgtagacagagaagactcttgggtttctgataggcactgactctctgtcccttgggctgttttcctaccctcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcagggtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgattaatttcacttatttcatttttttgtccaggtgtgtaagaaggttcctgaggctctacagatagggagcacttgtttattttacaaagagtacatgggaaaagagaaaagcaagggaaccgtacaaggcattaatgggtgacacttctacctccaaagagcagaaattatcaagaactcttgatacaaagataatactggcactgcagaggttctagggaagacctcaaccctaagacatagcctcaagggtaatgctacgattaaactccaacaattactgagaaaataatgtgctcaattaaaggcataatgattactcaagacaatgttatgttgtctttcttcctccttcctttgcctgcacattgtagcccataatactataccccatcaagtgttcctgctccaagaaatagcttcctcctcttacttgccccagaacatctctgtaaagaatttcctcttatcttcccatatttcagtcaagattcattgctcacgtattacttgtgacctctcttgaccccagccacaataaacttctctatactacccaaaaaatctttccaaaccctcccccacaccattttttatatttttatatttttcttatttatttcatgcacacacacacactccgtgctttataagcaattctgcctattctctaccttcttacatgcctactgtgcctcatattaaattcatcaatgggcagaaagaaaatatttattcaagaaaacagtgaatgaatgaacgaatgagtaaatgagtaaatgaaggaatgattattccttgctttagaacttctggaattagaggacaatattaataataccatcgcacagtgtttctttgttgttaatgctacaacatacaaagaggaagcatgcagtaaacaaccgaacagttatttcctttctgatcataggagtaatatttttttccttgagcaccatttttgccataggtaaaattagaaggatttttagaactttctcagttgtatacatttttaaaaatctgtattatatgcatgttgattaattttaaacttacttgaatacctaaacagaatctgttgtttccttgtgtttgaaagtgctttcacagtaactctgtctgtactgccagaatatactgacaatgtgttatagttaactgttttgatcacaacattttgaattgactggcagcagaagctcttttatatccatgtgttttccttaagtcattatacatagtaggcactgagaactctttatatctgaataagatatttaggaaccactggtttacatatcagaagcagagctactcagggcattttggggaagatcactttcacattcctgagcatagggaagttctcataagagtaagatattaaaaggagatacttgtgtggtattcgaaagacagtaagagagattgtagaccttatgatcttgatagggaaaacaaactacattcctttctccaaaagtcaaaaaaaaagagcaaatatagcttactataccttctattcctacaccattagaagtagtcagtgagtctaggcaagatgttggccctaaaaatccaaataccagagaattcatgagaacatcacctggatgggacatgtgccgagcacacacaattactatatgctaggcattgctatcttcatattgaagatgaggaggtcaagagatgaaaaaagacttggcaccttgttgttatattaaaattatttgttagagtagagcttttgtaagagtctaggagtgtgggagctaaatgatgatacacatggacacaaaaaatagatcaacagacacccaggcctacttgagggttgagggtgggaagagggagacgatgaaaaagaacctattgggtattaagttcatcactgagtgatgaaataatctgtacatcaagacccagtgatatgcaatttacctatataacttgtacatgtacccccaaatttaaaatgaaagttaaaacaaagtataggaatggaattaattcctcaagatttggctttaattttatttgataatttatcaaatggttgtttttcttttctcactatggcgttgctttataaactatgttcagtatgtctgaatgaaagggtgtgtgtgtgtgtgaaagagagggagagaggaagggaagagaggacgtaataatgtgaatttgagttcatgaaaatttttcaataaaataatttaatgtcaggagaattaagcctaatagtctcctaaatcatccatctcttgagcttcagagcagtcctctgaattaatgcctacatgtttgtaaagggtgttcagactgaagccaagattctacctctaaagagatgcaatctcaaatttatctgaagactgtacctctgctctccataaattgacaccatggcccacttaatgaggttaaaaaaaagctaattctgaatgaaaatctgagcccagtggaggaaatattaatgaacaaggtgcagactgaaatataaatttttctgtaataattatgcatatactttagcaaagttctgtctatgttgactttattgctttttggtaagaaatacaactttttaaagtgaactaaactatcctatttccaaactattttgtgtgtgtgcggtttgtttctatgggttctggttttcttggagcatttttatttcattttaattaattaattctgagagctgctgagttgtgtttactgagagattgtgtatctgcgagagaagtctgtagcaagtagctagactgtgcttgacctaggaacatatacagtagattgctaaaatgtctcacttggggaattttagactaaacagtagagcatgtataaaaatactctagtcaagtgctgcttttgaaacaaatgataaaaccacactcccatagatgagtgtcatgattttcatggaggaagttaatattcatcctctaagtatacccagactagggccattctgatataaaacattaggacttaagaaagattaatagactggagtaaaggaaatggacctctgtctctctcgctgtctcttttttgaggacttgtgtgtgtgtgtgtgtgtgtgtgtgtgtgtgtgttgtggtcagtggggctggaataaaagtagaatagacctgcacctgctgtggcatccattcacagagtagaagcaagctcacaatagtgaagatgtcagtaagcttgaatagtttttcaggaactttgaatgctgatttagatttgaaactgaggctctgaccataaccaaatttgcactatttattgcttcttgaaacttatttgcctggtatgcctgggcttttgatggtcttagtatagcttgcagccttgtccctgcagggtattatgggtaatagaaagaaaagtctgcgttacactctagtcacactaagtaactaccattggaaaagcaacccctgccttgaagccaggatgatggtatctgcagcagttgccaacacaagagaaggatccatagttcatcatttaaaaaagaaaacaaaatagaaaaaggaaaactatttctgagcataagaagttgtagggtaagtctttaagaaggtgacaatttctgccaatcaggatttcaaagctcttgctttgacaattttggtctttcagaatactataaatataacctatattataatttcataaagtctgtgcattttctttgacccaggatatttgcaaaagacatattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgcatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaaaggaaaaataaagaagtgaggctacatcaaactaaaaaatttccacacaaaaaacaaaacaatgaacaaatgaaaggtgaaccatgaaatggcatatttgcaaaccaaatatttcttaaatattttggttaatatccaaaatatataagaaacacagatgattcaataacaaacaaaaaattaaaaataggaaaataaaaaaattaaaaagaagaaaatcctgccatttatggcagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaattctagatatctaatgtacagcatcgtgactgtagttaattgtactgtaagtatatttaaaatttgcaaagagagtagatttttttttttttttagatggagttttgctcttgttgtccaggctggagtgcaatggcaagatcttggctcactgcaacctccgcctcctgggttcaagcaaatctcctgcctcagcctcccgagtagctgggattacaggcatgcgacaccatgcccagctaattttgtatttttagtagagacggggtttctccatgttggtcaggctgatccgcctgcctcggccacccaaagggctgggattacaggcgtgagccaccgggcctggccgagagtagatcttaaaagcatttaccacaagaaaaaggtaactatgtgagataatgggtatgttaattagcttgattgtggtaatcatttcacaaggtatacatatattaaaacatcatgttgtacaccttaaatatatacaatttttatttgtgaatgatacctcaataaagttgaagaataataaaaaagaatagacatcacatgaattaaaaaactaaaaaataaaaaaatgcatcttgatgattagaattgcattcttgatttttcagatacaaatatccatttgactgtttactcttttccaaaacaatacaataaattttagcactttatcttcattttccccttcccaatctataattatatatatatatattttagatattttgtatagttttactccctagattttctagtgttattattaaatagtgaagaaatgtttacacttatgtacaaaatgttttgcatgcttttcttcatttctaacattctctctaagtttattctatttttttctgattatccttaatattatctctttctgctggaaatacattgttacttttggtttatctaaaaatggcttcattttcttcattctaaaatcatgttaaattaataccactcatgtgtaagtaagatagtggaataaatagaaatccaaaaactaaatctcactaaaatataataatgtgatatataaaaatatagcttttaaatttagcttggaaataaaaaacaaacagtaattgaacaactatactttttgaaaagagtaaagtgaaatgcttaactgcatataccacaatcgattacacaattaggtgtgaaggtaaaattcagtcacgaaaaaactagaataaaaatatgggaagacatgtatataatcttagagataacactgttatttaattatcaacccaaagtagaaactatcaagggagaaataaattcagtcaacaataaaagcatttaagaagttattctaggctgggagcggtggctcacacctgcaattgcagcactttgggaggcctagacaggcggatcacgacgtcaggagttcaagatcagcctggccaacatagtgaaacctcatcgctactaaaaatataaaaacttagcctggcgtggtggcaggcatgtgtaatcccagcaatttgggaggctgaggcaggagaatcgcttgatcctgggaggcagaggttgcagtgagccaagattgtgccactgcattccagcccaggtgacagcatgagactccgtcacaaaaaaaaaagaaaaaaaaaaggggggggggagcggtggagccaagatgaccgaataggaacagctccagtctatagctcccatcgtgagtgacgcagaagacgggtgatttctgcatttccaactgaggtaccaggttcatctcacagggaagtgccaggcagtgggtgcaggacagtaggtgcagtgcactgtgcatgagccaaagcagggcgaggcatcacctcacccgggaagcacaaggggtcagggaattccctttcctagtcaaagaaaagggtgacagatggcacctggaaaatcgggtcactcccgccctaatactgcgctcttccaacaagcttaacaaatggcacaccaggagattatatcccatgcctggctcagagggtcctacgcccatggagcctcgctcattgctagcacagcagtctgaggtcaaactgcaaggtggcagtgaggctgggggaggggtgcccaccattgtccaggcttgagcaggtaaacaaagccgcctggaagctcgaactgggtggagcccaccacagctcaaggaggcctgcctgcctctgtaggctccacctctaggggcagggcacagacaaacaaaagacaacaagaacctctgcagacttaaatgtccctgtctgacagctttgaagagagtagtggttctcccagcacatagcttcagatctgagaacaggcagactgcctcctcaagtgggtccctgacccccgagtagcctaactgggaggcatcccccagtaggggcagactgacacctcacatggctggtactcctctaagacaaaacttccagaggaatgatcaggcagcagcatttgcggttcaccaatatccactgttctgcagccaccgctgttgatacccaggaaaacagcttctggagtggacctccagtaaactccaacagacctgcagctgagggtcctgactgttagaaggaaaactaacaaacagaaaggacatccacaccaaaaacccatctgtacatcgccatcatcaaagaccaaaggtagataaaaccataaagatggggaaaaagcagagcagaaaaactggacactctaaaaatgagagtgcctctcctcctccaaagtaacgcagctcctcaccagcaatggaacaaagctgggcagagaatgactttgacgagttgagagaggaaggcttcagaagatcaaactactccaagctaaaggaggaagttcgaacaaacggcaaagaagtaaaaaactttgaaaaaaaattagatgaatggataactagaataaccaatgcacagaagtccttaaaggacctgatggagctgaaaaccaaggcaggagaactacgtgacaaatacacaagcctcagtaaccgatgagatcaactggaagaaagggtatcaatgacgaaagatgaaatgaatgaaatgaagcatgaagagaagtttagagaaaaaagaataaaaagaaacgaacaaagcctccaagaaatatgggactatgtgaaaagaccaaatctacatctaattggtgtagctgaaagtgatggggagaatggaaccaagttggaaaacactctgcaggatattatccaggagaacttccccaatctagcaaggcaagcccaaattcacattcaggaaatacagagaacgccacaaagatactcctagagaaaagcaactccaagacacataactgtcagattcaccaaagttgaaatgaaggaaaaaatgttaagggcagccagagagaaaggtcgggttacccacaaagggaagcccatcagactaacagctgatctatcggcagaaactctacaagccagaagaaagtgggggccaatattcaacattgttaaagaaaagaattttcaacccagaatttcatatccagccaaactaagcttcataagtgaaggagaaataaaatcctttacagacaagcaaatgctgagagattttgtcaccaccaggcctgccctacaagagctcctgaaggaagcactaaacatggaaaggaacaactagtatcagccactgcaaaaacatgccaaattgtaaagaccatcaaggctaggaagaaactgcatcaacgagcaaaataaccagctaacatcataatgacaggatcaaattcatacataacaatactcaccttaaatgtaaataggctaaatgctccaattaaaagacacagactggcaaattggataaggagtcaagacccatctgtgttctgtattcaggaaacccatctcacgtgcagagacacacataggctcgaaataaaaggatggaggaatatctaccaagcaaatggaaaacaaaaaaaggcaggggttgcaatcctagtctctgataaaacagattttaaaccaacaaagatcaaaagagacaaagaaggccattacataatggcaaagggatctattcaagaagaagaactaactatactaaatatatatgcacccaatacaggagcacccagattcataaaacaagtcctgagtgacctacaaagagacttagatgcccacacaataataatgggagactttaacaccccactgtcaacattagacagatcaacgagacagaaagttaacaaggatatccaggaattggactcagctctgcaccaagcagacctaatagacatctacagaactctccaccccaaatcaacagaatatacattcttttcagcaccacaccacacctattccaaaactgaccacatagttggaagtaaagctctcctcagcaaatgtaaaagaacagaaactataacaaactgtctctcagaccacagtgcaatcaaactagaactcaggattaagaaactcactcaaaaccactcagctacatggaaactgaacagcctgctcctgaatgactactgggtacataacaaaatgaaggcagaaataaagatgttctttgaaaccaacgagaacaaagacacaacacaccagaatctctgagacacattcaaagcagtgtgtagagggaaatttatagcactaaatgcccacaagggaaagcaggaaagatctaaaattgacaccctaacatcacaattaaaaaactagagaagcaggagcaaacacattcaaaagctaacagaagacaagaaataactaagatcagagcagaagtgaaggacatagagacacaaaaaaacccttcaaaaaaatcaatgaatccagaagctgtttttttgaaaagatcaacaaaattgatagactgctagcaagactaataaagaagaaaagagagaagaatcaaatagacgcaataaaaaatgacacggggtatcaccactgatcccacagaaatacaaactaccgtcagagaatactataaacacctctacgcaaataaactagaaaatctagaagaaatggataaattcctcgacacatacactctgccaagactaaaccaggaagaagttgtatctctgaatagaccaataacaggctctgaaattgaggcaataattaatagcttatcaaccaaaaaaagtccgggaccagtaggattcatagccgaattctaccagaggtacaaggaggagctggtaccattccttctgaaactattccaatcaatagaaaaagagggaatcctccctaactcattttatgaggccagcatcatcctgataccaaagcctgacagagacacaacaaaaaaagagaatgttacaccaatatccttgatgaacattgatgcaaaaatcctcaataaaatactggcaaactgatccaccatgatcaagtgggcttcatccctgccatgcaaggctggttcaacatacgaaaatcaataaacataatccagcatataaacagaaccaaagacacaaaccatatgattatctcaatagatgcagaaaaggcctttgacaaaattcaacaacgcttcatgctaaaaactctcaataaattaggtattgatgggacatatctcaaaataataagagctatctatgacaaacccacagccaatatcatactgagtggacaaaaactggaagcattccctttgaaaactggcacaaggcagggatgccctctctcaccactcctattcaacatagtgttgtaagttctggccagggcaatcaggcaggagaaggaaataaagggcattcaattaggaaaagaggaagtgaaattgtccctgtttgcagatgacatgattgtatatctagaaaaccccattgtctcagcccaaaatctccttaagctgataagcaacttcagcaaagtctcaggatataaaatcagtgtgcaaaaatcacaagtattcctatgcaccaataacagacaaacagagagccaaatcatgagtgaactcccattcacaattgcttcaaagagaataaaatacctaggaatccaacttacaagggatgtgaaggacctcttcaaggagaactacaaaccactgctcaatgaaataaaagaggatacaaacaaatggaagaacattccatgctcatgggtaggaagaatcaatatcgtgaaaatggtcatactgcccaaggtaatttatagattcaatgccatccccatcaagctaccaatgactttcttcacagaactggaaaaaactactttaaagttcatatggaaccaaaaaagagcccacatcaccaaggcaatcctaagccaaaagaacaaagctggaggcatcacgctacctgacttcaaactatactacaatgctacggtaaccaaaacagcatggtactggtaccaaaacagagatctagaccaatggaacagaacagagccctcagaaataatgccgcatatctacaactatctgatctttgacaaacctgagagaaacaagcaatggggaaaggattccctatttaataaatggtgctgggaaaactggctagccatatgtagaaagctgaaactggatcccttccttacaccttatacaaaaattaattcaagatggattaaagacttacatgttagacctaaaaccataaaaaccctagaaaaaaacctaggcaataccattcaggacataggcatgggcaaggacttcatgtctaaaacaccaaaagcaatggcaacaaaagacaaaatggacaaacgggatctaattaaactaaagagcttctgcacagctaaagaaactaccatcagagtgaacaggcaacctacaaaatgggagaaaatttttgcaatctactcatctgacaaagggctaatatccagaatctacaatgaactcaaacaaatttacaagaaaaaacaaacaaccccatcaaaaagtgggcaaaggatatgaacagacacttcgcaaaagaagacatttatgtaatcaaaaaacacatgaaaaaatgctcatcatcactagccatcagagaaatgcaaatcaaaaccacaatgagataccatctcacaccagttagaatggcgatcattaaaaagtcaggaaacaacaggtgctggagaggatgtggagaaacaggaacaacttttacactgttggtgggactgtaaactagttcaaccattgcggaagtcagtgtggcaattcctcaggaatctagaactagaaataccatttgacccagccatcccattactgggtacatacccaaaggattataaatcatgctgctataaagacacatgcacacgtatgtttattgcagcactattcacaatagcaaagacttggaaccaacccaaatgtccaacaacgatagactggattaagaaaatgtggcacatatacaccatggaatactatgcagccataaaaaatgatgagttcatgtcctttgtagggacatggatgaagctggaaactatcattctcagcaaactatcacaaggagaataaaccaaacaccgcatgttctcactcataggtgggaattgaacaatgagaacacatggacacatgaagaggaacatcacactctggggactgttatggggtggggggcaggggcagggatagcactaggagatatacctaatgctaaatgacgagttaatgggtgcagcacaccaacatggcacatgtatacatatataacaaacctgcatgttgtgcacatgtaccctaaaacttgaagtataataataaaaaaaagttatcctattaaaactgatctcacacatccgtagagccattatcaagtctttctctttgaaatagacagaaatttagtgttttctcagtcagttaac